(19) United States
(12) Patent Application Publication
(10) Pub. No.: US 2018/0117355 A1
(43) Pub. Date: May 3, 2018

(54) DEVICES AND METHODS FOR PHOTOTHERAPY

(71) Applicant: KLOX TECHNOLOGIES INC., Laval (CA)

(72) Inventors: Nikolaos LOUPIS, Athens (GR); Remigio PIERGALLINI, San Benedetto Del Tronto (IT); Eric DESROSIERS, Outremont (CA)

(21) Appl. No.: 15/571,874

(22) PCT Filed: May 12, 2016
(86) PCT No.: PCT/CA2016/050544

§ 371 Date: Nov. 6, 2017

Related U.S. Application Data
(60) Provisional application No. 62/160,120, filed on May 12, 2015.

(51) Int. Cl. A61N 5/06

(52) U.S. Cl. CPC A61N 5/062; A61N 5/0603; A61N 2005/005; A61N 5/0616; A61N 5/0624

(57) ABSTRACT

A phototherapeutic device to perform a phototherapy treatment on a subject for healthcare including medical and/or dental, cosmetic, and/or other purposes. The phototherapeutic device comprises a lamp to emit light for treatment of the subject. The phototherapeutic device may also comprise a control system for controlling operation of the lamp; a cooling system for cooling the lamp; and a user interface for use by a user of the phototherapeutic device. The phototherapeutic device may also have various features to facilitate its use and/or treatment of the subject, such as, for example, an enhanced mobility of its lamp and/or an enhanced capability to provide feedback and/or other information to its user.

### CROSS-REFERENCE TO RELATED APPLICATIONS

This application claims the benefit of and priority to U.S. provisional patent application No. 62/160,120, filed May 12, 2015, the content of which is herein incorporated in its entirety by reference.

### FIELD

This disclosure relates generally to phototherapy and, more particularly, to devices and methods for treatment of subjects (e.g., humans and animals) using controlled application of light.

### BACKGROUND

Phototherapy is now used for a variety of healthcare (including medical and dental), cosmetic, and other purposes. For example, phototherapy can be used to promote wound healing, whiten teeth, kill bacteria or viruses, promote skin rejuvenation, treat skin conditions such as acne, even skin tone, reduce or prevent scarring, or the like.

Various phototherapeutic devices have been developed, however some drawbacks have been observed with these devices. For examples, these phototherapeutic devices are not readily transportable and readily adaptable to various treatment settings. Therefore, in some treatment settings where the phototherapeutic device is not readily adaptable to external factors, such as, for example, position of the patient, accessibility of the treatment site, or the like, the delivery of the phototherapy may not constant and/or may not be optimal.

As such, there remains a need for improvements to address these as well as other drawbacks and to facilitate use of and/or treatment of subjects with such phototherapeutic devices.

### SUMMARY OF DISCLOSURE

According to various aspects of the present disclosure, there is provided a phototherapeutic device to perform a phototherapy treatment on a subject for healthcare (including medical and/or dental), cosmetic, and/or other purposes. The phototherapeutic device comprises a lamp to emit light for treating the subject. The phototherapeutic device may also comprise a support for supporting the lamp on a floor or other surface. The phototherapeutic device may have various features to facilitate its use and/or treatment of the subject, such as, for example, an enhanced mobility of its lamp and/or an enhanced capability to provide feedback and/or other information to its user.

According to various aspects of the present disclosure, there is provided a phototherapeutic device comprising connection means; and a lamp connected to the connection means, the lamp being detachably connected to the connection means such that the lamp is selectively disconnectable and removable from the connection means to be used independently from the connection means and reconnectable to the connection means to be used while supported by the connection means, the lamp comprising a light source; wherein the light source emits light for phototherapy of a subject.

According to various aspects of the present disclosure, there is provided a phototherapeutic device comprising a lamp, the lamp comprising: a light source; a control system for controlling operation of the lamp; a cooling system for cooling the lamp; and a user interface for use by a user of the phototherapeutic device; wherein the light source emits light for phototherapy of a subject.

According to various aspects of the present disclosure, there is provided the use of a phototherapeutic device as defined herein in phototherapy of a subject in need thereof.

According to various aspects of the present disclosure, there is provided the use of a phototherapy device as defined herein in a method of treatment of a subject, wherein the method of treatment comprises phototherapy.

According to various aspects of the present disclosure, there is provided a method for phototherapy of a treatment site on a subject, said method comprising: applying a photoactivatable composition to the treatment site; and exposing the applied photoactivatable composition to light emitted by a phototherapeutic device as defined herein to activate the photoactivatable composition; wherein activation of the photoactivatable composition allows treatment of the treatment site.

According to various aspects of the present disclosure, there is provided a method for phototherapy of a treatment site on a subject, said method comprising: mounting a photoactivatable composition to a phototherapeutic device as defined herein; and exposing the treatment site to light emitted by the phototherapeutic device to activate the photoactivatable composition; wherein activation of the photoactivatable composition allows treatment of the treatment site.

According to various aspects of the present disclosure, there is provided a method for phototherapy of a treatment site on a subject, said method comprising: mounting a first photoactivatable composition to a phototherapeutic device as defined herein; applying a second photoactivatable composition to the treatment site; and exposing the treatment site to light emitted by the phototherapeutic device to activate the first and the second photoactivatable compositions; wherein activation of the first and the second photoactivatable compositions allows treatment of the treatment site.

According to various aspects of the present disclosure, there is provided a system for phototherapy of a subject, the system comprising at least one photoactivatable composition and at least one phototherapeutic device as defined herein.

According to various aspects of the present disclosure, there is provided a kit for phototherapy of a subject, the kit comprising a phototherapeutic device as defined herein; and instructions for use of the phototherapeutic device for phototherapy of a subject.

According to various aspects of the present disclosure, there is provided a kit for phototherapy of a subject, the kit comprising: a phototherapeutic device as defined herein; and a connection means.

These and other aspects of the present disclosure will now become apparent to those skilled in the art upon review of the following description of embodiments of the present disclosure in conjunction with the accompanying drawings.

### BRIEF DESCRIPTION OF THE DRAWINGS

A detailed description of embodiments of the present disclosure is provided below, by way of example only, with reference to the accompanying drawings, in which:

FIG. 1 shows an example of a phototherapeutic device for performing a phototherapy treatment on a subject in accordance with an embodiment of the present disclosure;

FIGS. 2A, 2B and 3 show perspective and side views of implementations of the phototherapeutic device;

FIG. 4 shows components of a lamp of the phototherapeutic device;

FIGS. 5A and 5B show top and bottom views of the lamp;

FIG. 6 shows an irradiated area exposed to light emitted by the lamp;

FIG. 7 shows components of the lamp;

FIG. 8 shows a light source of the lamp;

FIG. 9 shows a control system of the lamp;

FIG. 10 shows an interface of the control system of the lamp;

FIGS. 11 and 12 show a user interface of the lamp;

FIG. 13 shows a controller of the control system of the lamp;

FIG. 14 shows sensors of the control system of the lamp;

FIG. 15 shows a power supply of the control system of the lamp;

FIG. 16 shows a cooling system of the lamp;

FIGS. 17 to 19 and 20A-B show components of a support of the phototherapeutic device;

FIGS. 21A-B show a lamp-connecting system of the phototherapeutic device to detachably connect the lamp to the support;

FIGS. 22A to 22F show various positions of the lamp by adjusting the support;

FIG. 23 shows a photoactivatable composition that can be exposed to the light emitted by the phototherapeutic device for treating the subject;

FIG. 24 shows a variant in which the photoactivatable composition is implemented as a solid object;

FIG. 25 shows a holder of the phototherapeutic device for holding the photoactivatable composition;

FIG. 26 shows a variant in which the phototherapeutic device is used in combination with the photoactivatable composition and another, separate photoactivatable composition;

FIG. 27 shows a variant in which the lamp is one of a plurality of different lamps that can be connected to the support;

FIG. 28 shows a camera of the phototherapeutic device; and

FIGS. 29A-C show a casing for the phototherapeutic device in accordance with an embodiment of the present disclosure.

It is to be expressly understood that the description and drawings are only for the purpose of illustrating certain embodiments of the present disclosure and are an aid for understanding. They are not intended to be a definition of the limits of the disclosure.

### DETAILED DESCRIPTION OF EMBODIMENTS

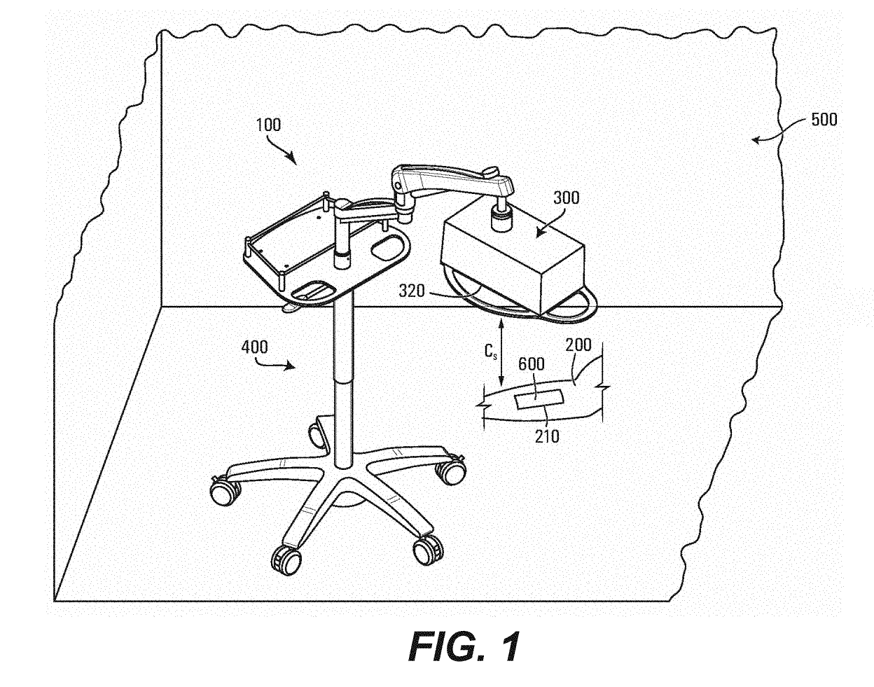

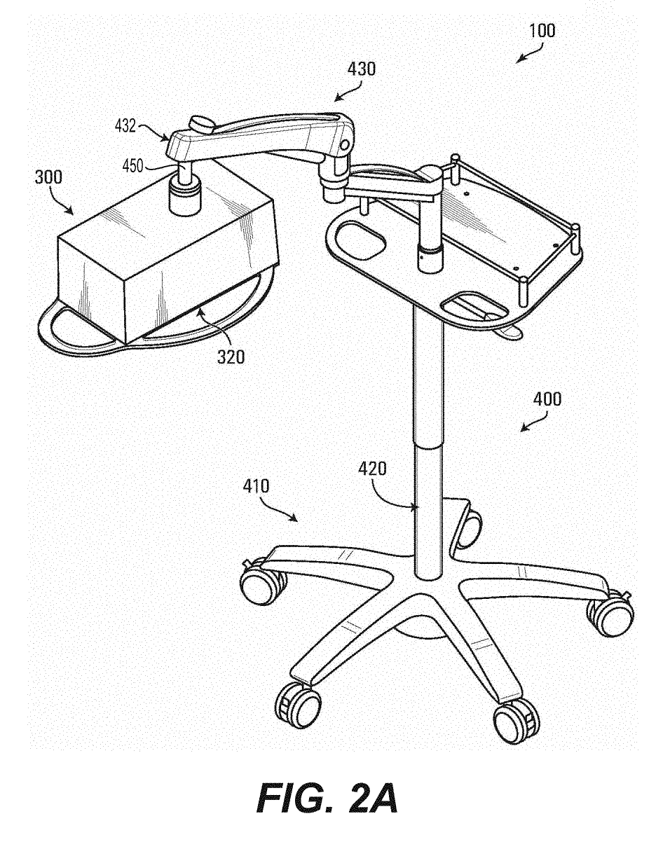

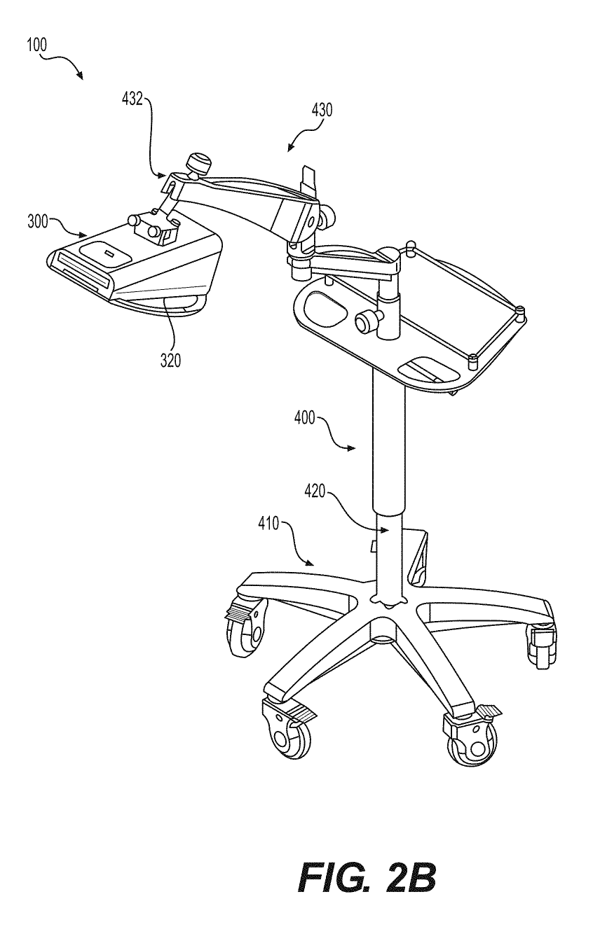

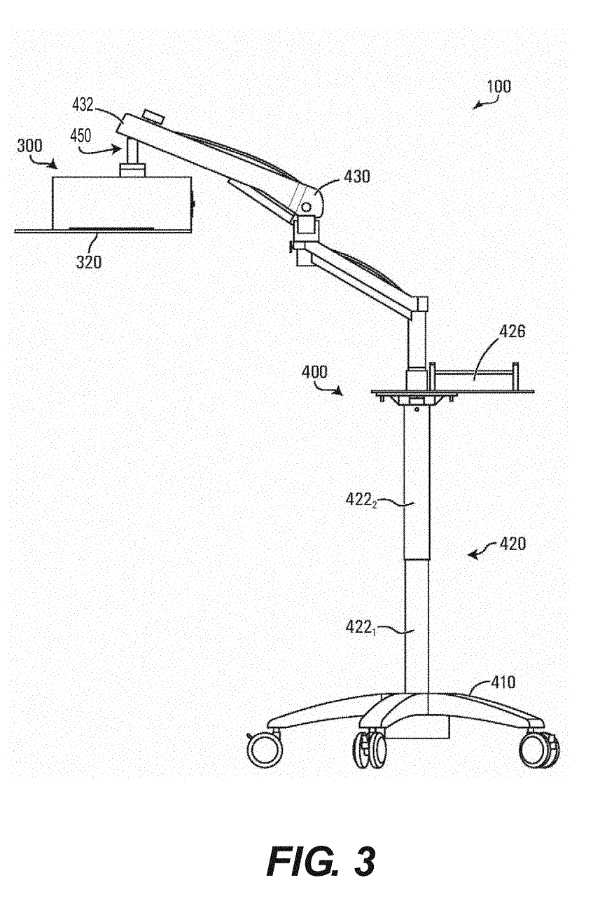

FIGS. 1 to 6 illustrate an example of a phototherapeutic device 100 for performing a phototherapy treatment on a subject 200 in accordance with an embodiment of the present disclosure. In this example, the subject 200 is a human. The phototherapeutic device 100 comprises a lamp 300 to emit light for treating the subject 200, including a treatment site 210 of the subject 200. In this embodiment, the phototherapeutic device 100 also comprises a support 400 for supporting the lamp 300 on a surface 500. In this example of implementation, in order to implement the phototherapeutic treatment, the phototherapeutic device 100 may be used in combination with a photoactivatable composition 600, of which at least a part may be applied on the subject 200.

The phototherapeutic device 100 can be used for healthcare (including medical and/or dental), cosmetic, and/or other purposes. For instance, in various examples of implementation, the phototherapeutic device 100 can be used, for example, to:
treat a wound (e.g., to help healing);
provide an antibacterial, antiviral, antifungal and/or anti-parasitic treatment;
treat an inflammation;
treat skin (e.g., rejuvenate the skin's appearance; treat a skin condition such as acne, psoriasis, rosacea, dermatitis, eczema, or the like) and soft tissues;
prevent the appearance of a skin condition such as acne, psoriasis, rosacea, dermatitis, eczema, or the like;
even the skin's tone;
reduce, eliminate or prevent scarring; and
prevent and/or treat oral and/or dental conditions.

The phototherapeutic device 100 can be used by a user, who may be, for example, a healthcare practitioner such as for example, a clinician (e.g., a physician, a nurse, a medical technician, a dentist, a dental assistant, or any other healthcare practitioner), a cosmetic practitioner, the subject 200 himself/herself, or any other suitable individual. The phototherapeutic device 100 may be used in a residence, a healthcare facility (e.g., a hospital, a medical or dental clinic, or the like), a cosmetic facility, and/or any other suitable setting.

As further discussed below, in some embodiments, the phototherapeutic device 100 has various features to facilitate its use and/or treatment of the subject 200, such as, for example, an enhanced mobility of the lamp 300 and/or an enhanced capability to provide feedback and/or other information to its user.

In some embodiments, as will be described below, the phototherapeutic device 100 is the lamp 300 itself.

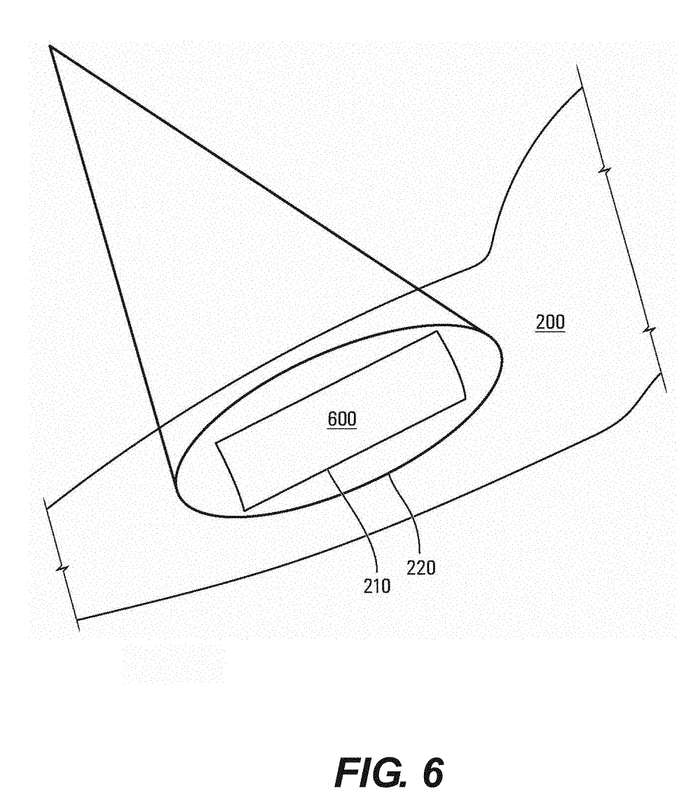

The lamp 300 comprises a light source 320 to emit light for the phototherapy treatment on the subject 200. As shown in FIG. 6, in this example, the light emitted by the light source 320 irradiates an irradiated area 220 which intersects the treatment site 210 of the subject 200 that is covered by the photoactivatable composition 600.

In some embodiments, the phototherapeutic device 100 comprises a support 400 which is configured as a stand and the surface 500 is a floor such that the phototherapeutic device 100 is positionable on the surface 500. In this example, the support 400 is configured such that the phototherapeutic device 100 is movable (e.g., rollable) on the surface 500. In other embodiments, the surface 500 may be a wall, an item of furniture, or any other suitable surface on which the lamp 300 can be supported.

More particularly, in this embodiment, the support 400 comprises a base 410 resting on the surface 500, a post 420 extending upwardly from the base 410, and an arm 430 extending from the post 420. The arm 430 is supported by the post 420 and configured for carrying the lamp 300 at a distal end portion 432 of the arm 430. The lamp 300 is movable relative to the subject 200 as the arm 430 is moved.

In this embodiment, the lamp 300 is detachably connected to the support 400 such that the lamp 300 is selectively disconnectable and removable from the support 400 to be used independently from the support 400 and reconnectable to the support 400 to be used while supported by the support 400. More specifically, in this embodiment, the lamp 300 contains within itself all those components used for its operation, which are discussed in greater detail below. Accordingly, in this embodiment, the lamp 300 may be removable from the support 400 and usable as a stand-alone device by the user.

In such implementations, the lamp 300 may be selectively attached to and detached from the support 400 by connection means such as for example a lamp-connecting system 450 that may releasably connect to the arm 430 and the lamp 300 and that may allow the lamp 300 to be temporarily attached to the surface 500 or to an item of furniture such as, but not limited to, a healthcare facility/hospital bed, and positionally adjustable while temporarily attached to the surface 500 or to the item of the furniture or to other healthcare items.

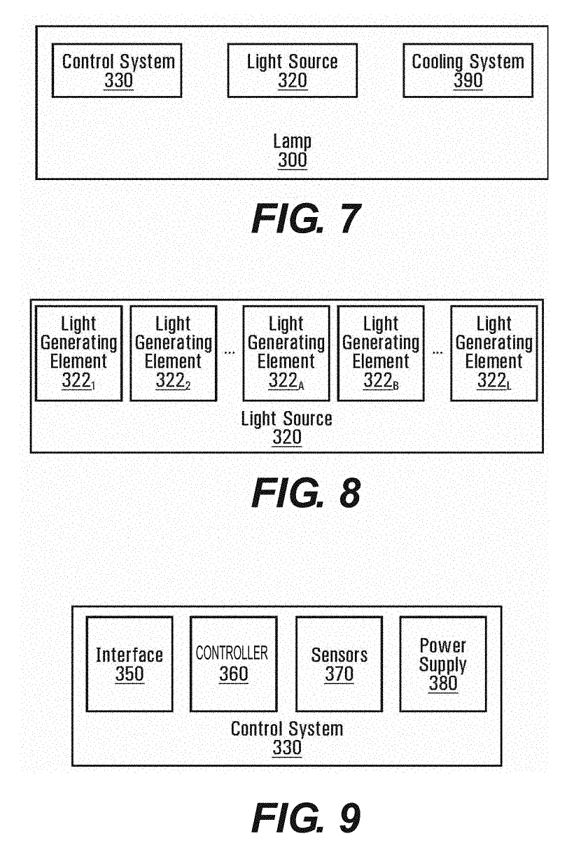

With additional reference to FIG. 7, in this embodiment, the lamp 300 comprises a control system 330 for controlling operation of the light source 320 and a frame 310 supporting various components of the lamp 300, including the light source 320 and the control system 330. Also, in this embodiment, the lamp 300 comprises a cooling system 390 to manage heat generated by the light source 320 and the control system 330.

In this embodiment, the frame 310 comprises a housing 312 defining a cavity 319 that houses various components of the lamp 300, including at least part of the light source 320, the control system 330, and the cooling system 390. The frame 310 also comprise handles 318₁-318₃ for allowing the user to handle (e.g., move and/or hold) the lamp 300. The frame 310 may be composed of one or more of a variety of materials, including different varieties of plastics and metals, or any combination thereof.

The housing 312 may be implemented in any suitable way, taking on any suitable shape. The housing 312 comprises a periphery 314 which includes a top surface 315, a bottom surface 311, and lateral surfaces 317₁-317₄. For example, in some embodiments, the housing 312 may take on the shape of a rectangular box with a length, a width, and a height, where the magnitude of the length is larger than the magnitude of the width. In other embodiments, the housing 312 may be a cuneiform rectangular box, or may define a generally semi-spherical or semi-ovoidal shape. In some embodiments, the housing 312 may taper in a lengthwise direction or in a widthwise direction of the lamp 300. Many other possible shapes of the housing 312 are possible. The housing 312 may be composed of one or more of a variety of materials, including different varieties of plastics and metals, or any combination thereof. In some embodiments, the housing 312 may be wholly formed as a one-piece component, whereas in some other embodiments, it may be composed of a plurality of pieces fastened together (e.g., using an adhesive and/or one or more mechanical fasteners such as screws, bolts, rivet, or the like; by welding, or the like).

In this embodiment, the cavity 319 of the housing 312 in which are housed various components of the lamp 300 tapers in the longitudinal direction of the lamp. In some cases, this may help with airflow within the cavity 319 for convective heat transfer, or may be to accommodate cooling systems, the details of which are discussed later.

The handles 318₁-318₃ may facilitate handling of the lamp 300 by the user. Notably, in this example, the handles 318₁-318₃ allow the user to handle the lamp 300 without obstructing a light transmission path from the lamp 300 to the subject 200. In this embodiment, each of the handles 318₁-318₃ is connected to the housing 312. This connection may be effected by way of an adhesive, one or more mechanical fasteners, a weld, an interlocking mechanism, or the like. In some cases, one or more of the handles 318₁-318₃ may be wholly formed with the housing 312 (i.e., the housing 312 and one or more of the handles 318₁-318₃ may be manufactured as a one-piece element). One or more of the handles 318₁-318₃ may be located on one or more of the lateral surfaces 317₁-317₄ of the housing 312. Alternatively, or in addition, one or more of the handles 318₁-318₃ may be located on the top surface 315 of the housing 312. Each of the handles 318₁-318₃ may have any suitable shape, such as a semi-circular shape, a semi-oval shape, or a semi-rectangular shape, or any other shape that lends itself to handling of the lamp 300 by the user. Although in this embodiment there are three handles, namely handles 318₁-318₃, embodiments of the lamp 300 with less than three handles, or more than three handles, may also be considered. For instance, in some embodiments, the lamp 300 may include only two handles, which may be located on opposite ones of the lateral surfaces 317₁-317₄ of the lamp 300.

The light generated by the light source 320 is emitted at a light-emitting side 302 of the lamp 300. The light source 320 may be implemented by any source of light that can emit light having certain characteristics desirable for the phototherapy treatment on the subject 200. In various examples of implementation, the light emitted by the light source 320 may include a pulsed light wave or a continuous light wave that may be spectrally concentrated (i.e., narrowband) or spectrally diffuse (i.e., broadband).

In some implementations, the light-emitting side 302 of the lamp 300 comprises a protection layer (not shown) for safety of the user and for protecting the light source 320 from any surrounding elements (e.g., microorganisms, particles, gases, liquids, solids, of the like) that may otherwise enter in contact with the light source 320. The protection layer also provides a surface that may be readily cleaned and/or sanitized. The protection layer is preferably optically transparent so as to allow the light emitted by the light source 320 to pass through the protection layer and avoid any heating during operation of the lamp 300. In some instances, the protection layer may be made of one or more thermoplastic polymers such as polycarbonates. The protection layer may cover the entirety of the light source and may be affixed on the light-emitting side 302 of the lamp 300 through any suitable securing means (e.g., screws).

With additional reference to FIG. 8, in this embodiment, the light source 320 comprises a plurality of light-generating elements 322₁-322_{L} that collectively emit the light for the phototherapy treatment on the subject 12. More particularly, in this embodiment, the light-generating elements 322₁-322_{L} are light-emitting diodes (LEDs).

The light emitted by the light source 320 may have any suitable characteristics for the phototherapy treatment on the subject 200. In this embodiment, the characteristics of the light emitted by the light source 320 may be selected based on the photoactivatable composition 600 with which the phototherapeutic device 100 may be used.

In some embodiments, the light-generating elements 322₁-322_{L} may be configured to each emit the same type of light.

In other embodiments, the light-generating elements 322₁-322_{L} may be split into two or more groups, with each group emitting the same type of light. In such embodiments, a therapeutic effect of the light emitted by a first group of the light-generating elements 322₁-322_{L} may be augmented and/or complemented by a therapeutic effect of another group of the light-generating elements 322₁-322_{L}. Here, the “types” of light may be differentiated based on a number of factors, including peak wavelength, average wavelength, bandwidth, intensity, or the like.

For example, in some embodiments, the light-generating elements 322₁-322_{L} may be split into two groups, including a first group of light-generating elements 322₁-322_{A} emitting light at a first peak wavelength and a second group of light-generating elements 322_{B}-322_{L} emitting light at a second peak wavelength. For instance, in some examples, the first peak wavelength may be about 430 to about 500 nm, about 440 to about 500 nm, about 450 to about 500 nm, about 430 to about 475 nm, about 435 to about 470 nm, about 440 nm to about 460 nm, about 440 nm, about 450 nm, about 460 nm, or about 470 nm, while the second peak wavelength may be of about 400 to about 500 nm, about 400 to about 475 nm, about 400 to about 450 nm, about 400 to about 430 nm, or about 410 to about 420 nm, or about 415 nm. In some examples of implementation, the first peak wavelength may be from about 410 to about 430 nm, and the second peak wavelength may be from about 440 to about 470 nm.

In some alternative embodiments, the second peak wavelength may be about 480 to about 760 nm, about 480 to about 700 nm, about 480 to about 650 nm, about 480 to about 620 nm, about 500 to about 700 nm, about 520 to about 700 nm, about 500 to about 660 nm, about 540 to about 640 nm, about 540 to about 580, about 500 to about 570 nm, about 570 to about 590 nm, about 590 to about 610 nm, about 610 to about 760 nm, or may be within the infrared range of the electromagnetic spectrum.

In some embodiments, the second peak wavelength may be longer than the first peak wavelength, and the distance between the first and the second peak wavelengths may be due to a Stoke's shift.

In certain embodiments, an average power density of the light emitted by the light source 320 and/or received by the treatment site 210, measured at a treatment distance (such as 5 cm, 10 cm, or any other suitable distance), is less than about 200 mW/cm², about 4 to about 75 mW/cm², about 15 to about 75 mW/cm², about 10 to about 200 mW/cm², about 10 to about 150 mW/cm², 20 to about 130 mW/cm², about 55 to about 130 mW/cm², about 90 to about 140 mW/cm², about 100 to about 140 mW/cm², or about 110 to about 135 mW/cm². In some embodiments, an average power density of the light emitted by the light source 320 is about 4 to about 75 mW/cm², about 10 to about 75 mW/cm², about 10 to about 85 mW/cm², about 15 to about 75 mW/cm², about 30 to about 70 mW/cm², about 40 mW/cm² to about 70 mW/cm², about 55 to about 85 mW/cm², or about 55 to about 65 mW/cm².

In certain embodiments, an average power density of the second group of light-generating elements 322₁-322_{L} is lower than an average power density of the first group of light-generating elements 322₁-322_{L}. For example, the average power density of the second group of light-generating elements 322₁-322_{L} may be about 0.1% to about 90%, about 0.1% to about 80%, about 0.1% to about 70%, about 0.1% to about 60%, about 0.% to about 50%, about 0.1% to about 40%, about 0.1% to about 30%, about 0.1% to about 20%, about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%, of the power density of the group of light-generating elements 322₁-322_{L}.

In some embodiments, the average power density of the light emitted by the second group may be less than about 75 mW/cm², less than about 50 mW/cm², less than about 10 mW/cm², less than about 5 mW/cm², less than about 2.5 mW/cm², or less than about 2 mW/cm². The power density at the peak wavelength can be from about 0.02 mW/cm²/nm to about 75 mW/cm²/nm, from about 0.02 to about 50 mW/cm²/nm, from about 0.02 to about 10 mW/cm²/nm, from about 0.02 to about 5 mW/cm²/nm, or from about 0.02 to about 10 mW/cm²/nm, about 0.005 to about 10 mW/cm²/nm, about 0.01 to 0.1 mW/cm²/nm, about 0.01 to about 2 mW/cm²/nm, about 0.01 to about 3 mW/cm²/nm, or about 0.5 to about 5 mW/cm²/nm.

In some other embodiments, the light-generating elements 322₁-322_{L} may be split into two, three, four, five, or more groups with each group emitting light with the same or different peak wavelength.

In certain embodiments, an average fluence emitted by the light source 320 and/or received by the treatment site 210, during a single phototherapy treatment procedure, is at least about 0.01 J/cm², or is more than about 4 J/cm², more than about 10 J/cm², more than about 15 J/cm², more than about 30 J/cm², more than about 50 J/cm², up to about 60 J/cm². In certain embodiments, the light source 320 is configured to emit light having a fluence, during a single treatment, of about 4 J/cm² to about 60 J/cm², about 10 J/cm² to about 60 J/cm², about 10 J/cm² to about 50 J/cm², about 10 J/cm² to about 40 J/cm², about 10 J/cm² to about 30 J/cm², about 20 J/cm² to about 40 J/cm², about 15 J/cm² to about 25 J/cm², or about 10 J/cm² to about 20 J/cm².

The light emitted by the light source 320 may have any other suitable characteristics for the phototherapy treatment on the subject 200 in other embodiments.

The light source 320 may be implemented in any other suitable way in other embodiments. For example, in other embodiments, the light source 320 may comprise a single light-generating element and/or may be implemented using any other type of light-generating element, such as, for instance, an incandescent light bulb, an electron-stimulated luminescence element such as an electroluminescent material (e.g., an electroluminescent wire or sheet), a field-induced polymer electroluminescent material, a gas-discharge bulb such as a fluorescent lamp, a cathode lamp, a neon or argon lamp, a plasma lamp, a xenon flash lamp, a high-intensity discharge lamp such as a metal-halide lamp, a diode laser, a fiber laser, an arc discharge, or the like.

The control system 330 is configured to control operation of the lamp 300, including the light source 320, and to provide information relating to operational parameters of the lamp 300 to the user of the lamp 300. For example, the control system 330 may turn on and off the light source 320, control (e.g., start, stop, modulate) emission of light (activate/deactivate certain LEDs, or the like) by the light source 320, control the cooling system 390 (e.g., turn on or off a fan, or the like), monitor parameters while the phototherapy treatment is being performed on the subject 200, cause information to be output to the user, and/or perform any other action. The control system 330 comprises suitable hardware and/or software (e.g., firmware) configured to implement its functionality.

With additional reference to FIG. 9, in this embodiment, the control system 330 comprises an interface 350, a controller 360, and one or more sensors 370. The control system 330 also comprises a power supply 380, which provides power to the lamp 300, including, in this case, to the light source 320 and the cooling system 390.

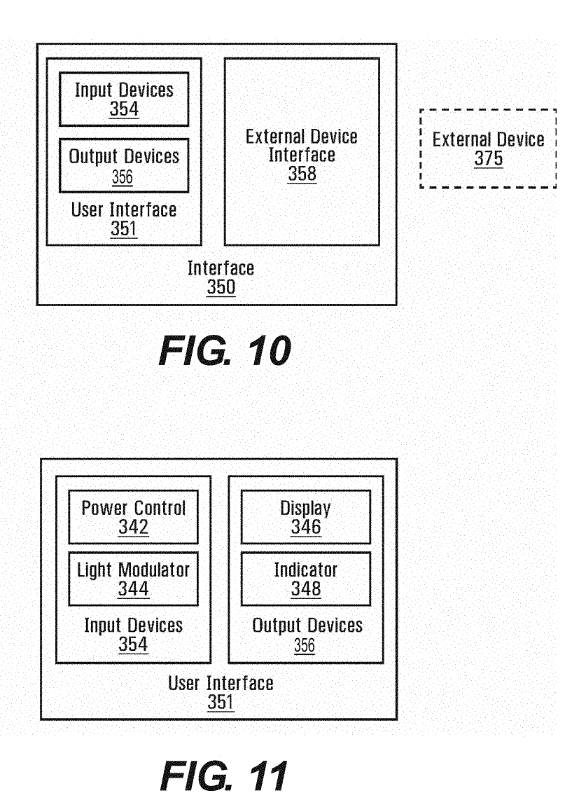

The interface 350 is configured to receive inputs, including commands, signals, and/or other information, and issue outputs, including commands, signals and/or other information. With additional reference to FIG. 10, in this embodiment, the interface 350 comprises a user interface 351 and an external device interface 358.

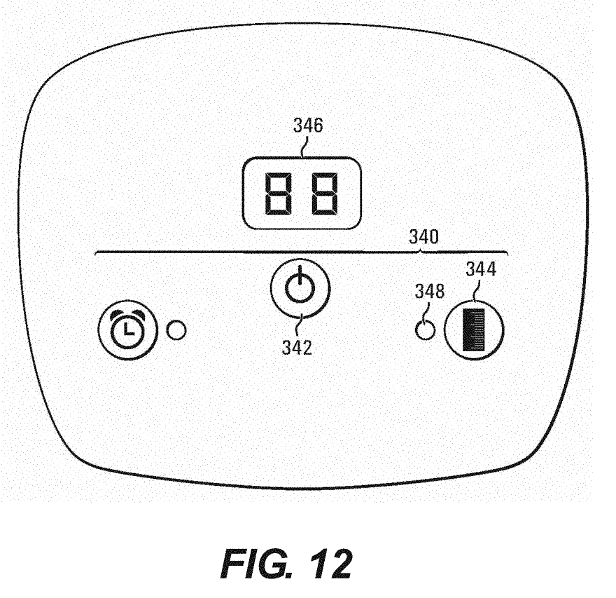

The user interface 351 is configured for receiving inputs from and outputting information to the user of the phototherapeutic device 100. To this end, in this example, the user interface 351 includes one or more input devices 354 and one or more output devices 356. With additional reference to FIGS. 11 and 12, in some embodiments, the input devices 354 may comprise one or more controls 340 for effecting control of the operation of the light source 320, including an on/off or power control 342, a light modulator 344 for modulating the output of the light source 320, and any other suitable controls. In some embodiments, the output devices 356 may include a display 346 which allows the user of the phototherapeutic device 100 to monitor one or more operational parameters of the lamp 300. The output devices 356 may also include an indicator 348, as discussed in greater detail below. The user interface 351 may include any number of additional and/or different input devices 354 and/or output devices 356.

The power control 342 is configured to control action of the power supply 380. When the power control 342 is in an “off” position, the power supply 380 is substantially prevented from providing power to the phototherapeutic device 100; conversely, when the power control 342 is in an “on” position, the power supply 380 is substantially permitted to provide power to the phototherapeutic device 100, thereby allowing the lamp 300 and its various components, including the light source 320 and the cooling system 390 to function.

The light modulator 344 may be configured to start or stop the light generation of one or more of the light-generating elements 322₁-322_{L}. Alternatively, or in addition, the light modulator 344 may also be configured to modulate the light generation of one or more of the light-generating elements 322₁-322_{L}. That is to say, the light modulator 344 may be configured to effect change in the operation of one or more of the light-generating elements 322₁-322_{L} so as to alter, among others, the average or instantaneous power density, average or instantaneous fluence, the average or peak wavelength, or the average or instantaneous bandwidth of the light emitted by one or more of the light-generating elements 322₁-322_{L}. In some embodiments, the light modulator 344 may be configured to effect change in the operation of all of the light-generating elements 322₁-322_{L} at substantially the same time. In other embodiments, the light modulator 344 may be configured to effect change in the operation of individual ones of the light-generating elements 322₁-322_{L} separately from other individual ones of the light-generating elements 322₁-322_{L}, or may be configured to effect change in the operation of the first group of the light-generating elements 322₁-322_{A} separately from the second group of the light generating elements light-generating elements 322_{B}-322_{L}.

The display 346 is configured to allow the user of the phototherapeutic device 100 to monitor one or more operational parameters of the lamp 300. The display 346 may only be active during the operation of the lamp 300, or may display information regardless of whether the lamp 300 is currently executing a phototherapeutic treatment procedure. The display 346 may be configured to display information relating to one or more parameters of the phototherapeutic device 100. In some embodiments, the display 346 may be configured to display an amount of time elapsed since the beginning of a phototherapy treatment procedure; alternatively, the display 346 may be configured to display an amount of time remaining until the end of a phototherapy treatment procedure having a fixed time duration. In some embodiments, the display 346 may also be configured to display information related to the operational parameters of the lamp 300, such as the average or instantaneous power density, average or instantaneous fluence, the average or peak wavelength or wavelengths, or the average or instantaneous bandwidth or bandwidths of the light emitted by the lamp 300, or any other suitable operational parameter. In some embodiments, the display 346 may display at most one such parameter of the phototherapeutic device 100 at any given time. In other embodiments, the display 346 may be configured to display two or more of the operational parameters of the phototherapeutic device 100. In embodiments where the display 346 is configured to not display all operational parameters simultaneously, one or more of the controls 340 of the user interface 351 may be configured to allow a user of the phototherapeutic device 100 to cycle through the displayable parameters. In FIG. 12, the display 346 is shown as being a numeric liquid-crystal display, but the display 346 may be implemented as any suitable data-displaying device, including more complex screens, touchscreens, and the like.

In this embodiment, the external device interface 358 allows an external device 375 (e.g., a computer such as a server, a laptop computer, a desktop computer, a tablet, or another computing device; a smartphone or another mobile communication device; a portable memory stick, or the like) to connect to the lamp 300. The external device interface 358 may allow the external device 375 to exchange (i.e., send and receive) various commands, signals, and/or other information with the interface 350, and more generally, with the lamp 300. Thus, in some cases, the lamp 300 may be controllable by the external device 375; that is to say, various operational parameters of the lamp 300 may be changed by the external device 375 communicating via the external device interface 358. The external device interface 358 may include wired connectivity, such as via one or more wired protocols including USB (2.0, 2.1, 3.0, or the like), e-SATA, Thunderbolt™, RS-232, Ethernet protocols, and the like. Alternatively, or in addition, the external device interface 358 may include wireless connectivity, such as via one or more wireless protocols including Bluetooth®, ZigBee®, IEEE 802.11 (a, b, g, n, ac, ax, or the like), one or more cellular technologies (GSM/LTE/HSPA/HSPA+), one or more NFC (near-field communication) protocols, and the like.

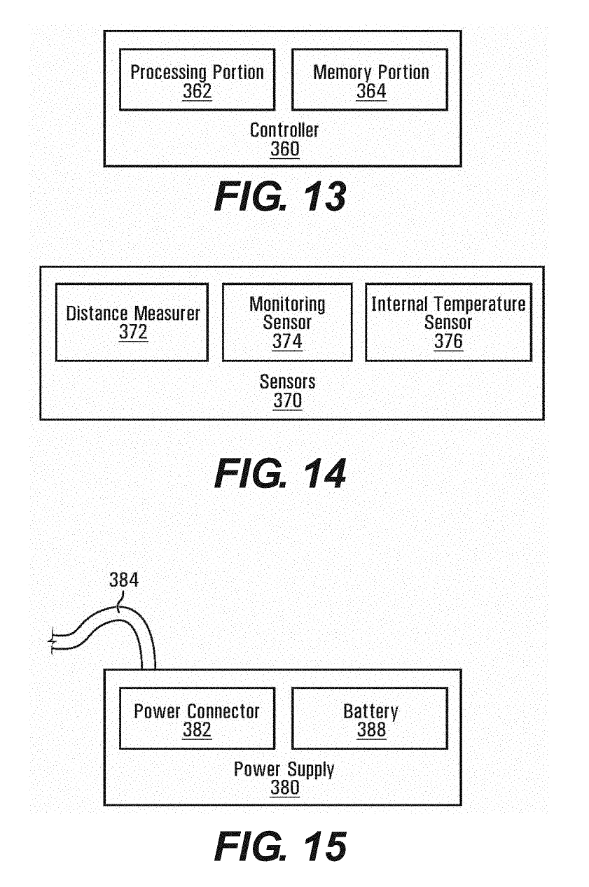

With additional reference to FIG. 13, the controller 360 is communicatively coupled to the interface 350 and comprises a processing portion 362 and a memory portion 364.

The processing portion 362 comprises one or more processors to perform processing operations that implement functionality of the controller 360. A processor of the processing portion 362 may be a general-purpose processor executing program code stored in the memory portion 364. Alternatively, a processor of the processing portion 362 may be a specific-purpose processor comprising one or more preprogrammed hardware or firmware elements (e.g., application-specific integrated circuits (ASICs), electrically erasable programmable read-only memories (EEPROMs) or other related elements).

The memory portion 364 comprises one or more memories for storing program code executed by the processing portion 362 and/or data used during operation of the processing portion 362. A memory of the memory portion 364 may be implemented as a semiconductor medium (including, e.g., a solid state memory), a magnetic storage medium, an optical storage medium, and/or any other suitable type of memory. A memory of the memory portion 364 may be read/writeable, read-only (ROM), and/or random-access (RAM). Of course, other implementations of the memory portion 364 are possible.

In this embodiment, the controller 360 comprises one or more printed circuit boards (PCBs) 355 implementing at least part of the processing portion 362 and the memory portion 364 and exchanging control signals with other components of the lamp 300, including with the light source 320 to control emission of light by the light source 320.

With additional reference to FIG. 14, in this embodiment, the control system 330 also comprises the one or more sensors 370 that are communicatively coupled to the interface 350 to provide to the controller 360 various information regarding a state of the lamp 300 and/or, more generally, conditions and progress of the phototherapy treatment.

In this embodiment, the sensors 370 include a distance measurer 372 to measure a distance Cₛ between the light-emitting side 302 of the lamp 300 and the subject 200 (as show in FIG. 1). This may help to have a proper or optimal spacing between the lamp 300 and the subject 200 for treatment purposes. The sensors 370 also include a monitoring sensor 374 for monitoring the progress of a phototherapy treatment procedure. Some embodiments of the control system 330 may comprise additional and/or different sensors. These may include infrared sensors, laser-based sensors, high-frequency radio-wave sensors, radar or other sound-based sensors, or any other suitable type of sensor, with further details being provided in the following paragraphs.

More particularly, in this embodiment, the distance measurer 372 is configured for sensing the distance Cₛ between the light-emitting side 302 of the lamp 300 and the subject 200. In some embodiments, the distance measurer 372 may comprise a wireless sensor that can sense the distance Cₛ between the light-emitting side 302 of the lamp 300 and the subject 200 without physically contacting the subject 200. The distance measurer 372 may be connected to the interface 350 either wiredly or wirelessly, and may implement one or more known range-finding techniques, including laser-based distance measurement, infrared-based distance measurement, visible-light-based distance measurement, or any other suitable range-finding technique. In some embodiments, the distance measurer 372 may be implemented as a short-range RFID system, where a reader (not pictured) located substantially within the lamp 300 may detect a response from a tag (not pictured) located substantially in contact with the subject 200 based on a message sent by the reader, where the distance Cₛ may be determined based on the response from the tag. Other implementations of the distance measurer 372 may also be possible.

The distance measurer 372 may be used to provide an indication of the distance Cₛ between the light-emitting side 302 of the lamp 300 and the subject 200 to the user. In some embodiments, the indicator 348 of the user interface 351 may convey the indication of the distance Cₛ between the light-emitting side 302 of the lamp 300 and the subject 200 to the user. In some embodiments, the indicator 348 may be a dedicated display separate from display 346; in other embodiments, the indicator 348 may be the display 346 of the user interface 351, and the display 346 may display the indication of the distance Cₛ along (e.g., simultaneously or not) with other information, as discussed above. In still other embodiments, the indicator 348 may be a visual indicator such as an LED or other light-source, which may be configured to vary a color or intensity of the light it emits to convey information. In still other embodiments, the indicator 348 may be a sound-emitting device, and may be configured to emit one or more sounds or patterns of sounds to convey information.

The indication of the distance Cₛ provided to the user may convey a value of the distance Cₛ (e.g., in centimeters, meters, inches, feet, or the like). In such embodiments, the indication of the distance Cₛ may be displayed on the display 346 as a numerical value.

Alternatively, or additionally, the indication of the distance Cₛ provided to the user may convey that the distance Cₛ is too large or too small relative to a predetermined reference distance for the treatment site 210 of the subject 200 (which may depend on the light emitted, type of treatment, or the like). For example, in some embodiments, a notification, such as a textual message (e.g., “lamp too close”, “lamp too far”, “lamp properly positioned”, or the like), a graphical icon (e.g., an arrow pointing towards or away from a human representation), and/or another message, may be conveyed on the display 346. As another example, in embodiments where the indicator 348 comprises a light, the indicator 348 may be configured to vary the color or intensity of light it outputs to convey the indication of the distance Cₛ to the user. For instance, the light emitted by the indicator 348 may be red if the light-emitting side 302 of the lamp 300 is too close to the subject 200, green if the light-emitting side 302 of the lamp 300 is an appropriate distance from the subject 200, and violet if the light-emitting side 302 of the lamp 300 is too far away from the subject 200. In some embodiments, the color displayed by the indicator may one of a plurality of colors, each associated with a specific distance (e.g.: red=<5 cm, orange=5 to 10 cm, or the like). In embodiments where the indicator 348 is configured to vary the intensity of the light it emits, the indicator may output a very bright light when the light-emitting side 302 of the lamp 300 is too close or too far from the subject 200, gradually dimming as the light-emitting side 302 of the lamp 300 approaches an appropriate distance from the subject 200. In embodiments where the indicator 348 is a sound-emitting device, the indicator 348 may be configured to emit louder sounds, or more frequent sounds, as the light-emitting side 302 of the lamp 300 approaches and appropriate distance from the subject 200. Other embodiments and implementations of the indicator 348 are also possible.

In some embodiments, the control system 330 may be configured to control the emission of light by the lamp 300 based on the distance Cₛ between the light-emitting side 302 of the lamp 300 and the subject 200. In the event the distance Cₛ is unsuitable for the treatment, be it too large or to small (relative to a predetermined reference distance), the control system 330 may take an action accordingly. For example, the control system 330 may be configured to convey an indication of the unsuitability of the distance Cₛ to the user, for example by way of the display 346 or the indicator 348. As another example, the control system 330 may be configured to decrease the intensity of the light emitted by the lamp 300 or to substantively turn off the lamp 300 in response to the distance Cₛ being unsuitable.

In this embodiment, the monitoring sensor 374 is configured for monitoring the irradiated area 220 which is irradiated by the light emitted by the lamp 300 of the phototherapeutic device 100 to provide an indication of a level of progress of the phototherapy treatment that may allow the controller 360 to control operation of the lamp 300 based on the indication of the level of progress provided by the monitoring sensor 374. In this example, the irradiated area 220 may intersect the photoactivatable composition 600 and/or the treatment site 210 of the subject 200, as discussed above. The monitoring sensor 374 may be an optical sensor, a thermal sensor, a camera, or any other suitable sensing mechanism, which in some embodiments may include suitable image processing capability (which may be inherent to the monitoring sensor 374 or distributed between the monitoring sensor 374 and the controller 360). Examples of ways in which the monitoring sensor 374 may be used are discussed later on.

In some embodiments, two or more sensors 370 of the control system 330, such as the distance measurer 372 and the monitoring sensor 374, may be implemented as the same sensor. In other embodiments, two or more sensors 370 of the control system 330, such as the distance measurer 372 and the monitoring sensor 374, may share at least some components.

With additional reference to FIG. 15, the power supply 380 is for supplying power to operate the lamp 300. In this embodiment, the power supply 380 comprises a power connector 382 for connecting to an external power source (not pictured). In this example, the power connector 382 is connectable to the external power source via a power cable 384 which may route or otherwise provide power from the external power source to the power supply 380. The power cable 384 may be any suitable cable, including, for example, a cable satisfying one or more of the IEC 60320 standards, such as the so-called IEC cord. In other examples, the power connector 382 may be an inductive charging interface which may be inductively coupled to a charging station (not pictured) and may be configured to convert an electromagnetic field generated by the charging station into electrical power supplied to the lamp 300.

In this embodiment, the power connector 382 may be located on the periphery 314 of the housing 312 of the lamp 300. In some instances, the power connector 382 may be located on a surface of the periphery 314 of the housing 312 where none of the handles 318₁-318₃ are located. The power connector 382 may be located at any other suitable location in other embodiments.

The power cable 384 comprises two ends and a length of cable therebetween. A first one of the ends of the power cable 384 is configured to connect to the power connector 382, and a second one of the ends of the power cable 384 is configured to connect to the external power source. The position assumed by the length of cable of the power cable 384 may not be specifically defined, though in some embodiments at least a portion of the length of cable may follow the support 400. For instance, in some embodiments, the power cable 384 may be configured to substantially run the length of the post 420 and/or the arm 430. In some embodiments, the post 420 and/or the arm may comprise one or more attachment devices (e.g., clips, clamps, or the like) which serve to secure the power cable 384 to the post 420 and/or to the arm 430. In some embodiments, a substantial portion of the power cable 384 may be positioned inside the post 420 and/or the arm 430, with a separate portion being exposed at a bottom portion of the post 420, at or near the point where the arm 430 connects to the post 420, or through the base 410.

In this embodiment, the power supply 380 includes a battery 388. The battery 388 may be implemented using any suitable battery technology, including lead-acid, nickel-cadmium, nickel-metal-hydride, lithium-ion (including lithium-polymer, lithium-iron-phosphate, lithium-titanate) and the like. The battery 388 may act as a primary power source for the lamp 300, such that the lamp 300 may be capable of operating for extended periods of time relying solely on the power provided by the battery 388, and may only need to be recharged by the external power source periodically. This may be useful, for instance, when the lamp 300 is used as a stand-alone device separate from the support 400. In other embodiments, the battery 388 may act as a backup power source, preventing the lamp 300 from turning off in the event of a failure of the external power source. In still further embodiments, the battery 388 may at times act as the primary power source and at other times act as the secondary power source.

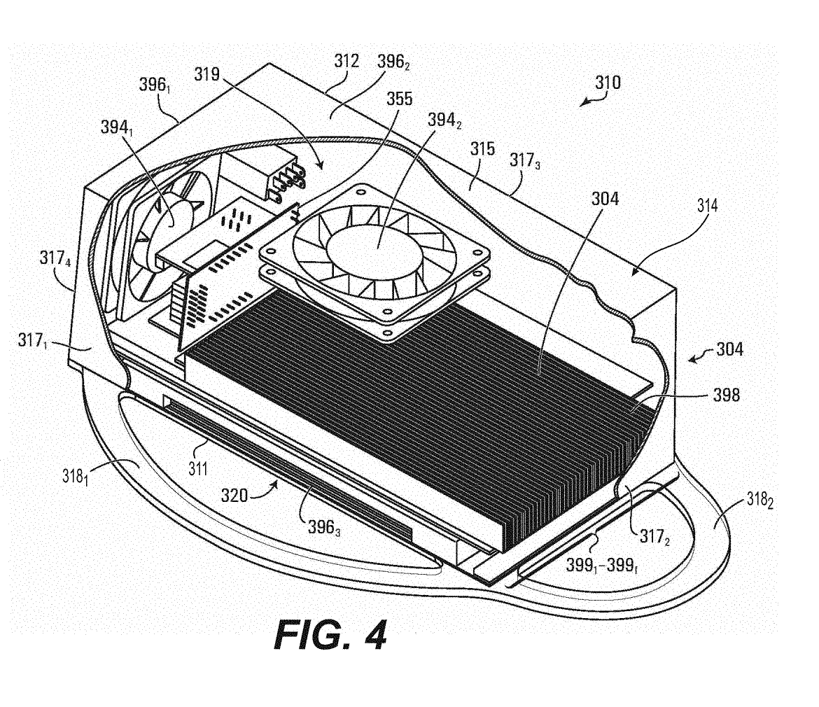

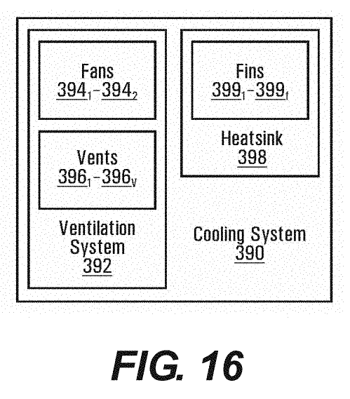

With reference to FIG. 4 and additional reference to FIG. 16, the cooling system 390 helps dissipate heat generated by the light source 320 and the control system 300. In this embodiment, the cooling system 390 comprises a ventilation system 392 and a heatsink 398 which may be exposed to air circulated by the ventilation system 392.

More particularly, in this embodiment, the heatsink 398 is adjacent to the light source 320 to conduct heat from the light source 320 and dissipate that heat by convection. In this example, the heatsink 398 comprises a plurality of fins 399₁-399_{f}, which are elongated in the longitudinal direction of the lamp 300 and reach toward a longitudinal end 304 of the lamp 300.

Also, in this embodiment, the ventilation system 392 comprises a plurality of fans 394₁-394₂ and a plurality of vents (or ventilation openings) 396₁-396ᵥ.

In this example, the fan 394₁ is configured to direct airflow toward at least part of the control system 330, which is implemented on the PCB 355. More specifically, by directing this airflow, the fan 394₁ may effect a dissipation of heat from the PCB 355 via convection. In this case, the fan 394₁ is located and exposed at the periphery 314 of the housing 312 adjacent to the PCB 355. Additionally, the fan 394₁ is positioned such that a projection of the fan 394₁ parallel to an axis of rotation of the fan 394₁ intersects the PCB 355. Also, in this example, the fan 394₂ is configured to direct airflow toward the heatsink 398 thereby cooling at least part of the heatsink 398. More specifically, by directing this airflow, the fan 394₂ may effect a dissipation of heat from the heatsink 398 via convection. In this case, the fan 394₂ is configured to direct the airflow along and/or between the fins 399₁-399_{f}.

In this embodiment, the fans 394₁-394₂ are oriented differently from one another. Put differently, their respective axis of rotation are transversal (i.e., nonparallel) to one another. In some such embodiments, the respective axis of rotation of each of fans 394₁-394₂ may be substantively perpendicular to one another. In some cases, the particular orientation of the fans 394₁-394₂ may induce a certain amount of turbulence, which may help to more efficiently dissipate heat by convection.

The vents 396₁-396ᵥ are configured to allow air to flow in and out of the housing 312 of the lamp 300. The vents 396₁-396ᵥ are located on the periphery 314 of the frame 310, and may be configured so as to permit airflow between the cavity 319 and an exterior environment in which the phototherapeutic device 100 operates. For example, in this embodiment, at least one of the vents 396₁-396ᵥ is located adjacent to the fan 394₁. Also, in this embodiment, at least one of the vent 396₁-396ᵥ is located at the longitudinal end 304 of the housing 312 where the fins 399₁-399_{f} terminate In some embodiments, each of the lateral surfaces 317₁-317₄ may comprise at least one vent. In other embodiments, only some of the lateral surfaces 317₁-317₄ may comprise at least one vent. In some embodiments, the vents 396₁-396ᵥ may only be located on those lateral surfaces which do not have any one of the handles 318₁-318₃. The vents 396₁-396ᵥ may be arranged in any other suitable manner in other embodiments.

In certain implementations of this embodiment, the housing 312 comprises means (not shown) for directing the flow of air exiting fan 394₁, such as by, but not limited to, air ventilation flaps and/or adjustable rings comprising one or more portals for directing the flow of the air exiting fan 394₁. The direction of the air ventilation flaps and/or portal-bearing rings may be adjustable (using, for example, a knob) so as to deflect the flow of air exiting fan 394₁ away from the subject (e.g., patient). The means for directing the flow of air exiting fan 394₁ may be mounted on top of fan 394₁ and may be affixed onto surface 317₄ of the housing 312.

The control system 330, and more specifically, the controller 360, is configured to control the fans 394₁-394₂. In some embodiments, the controller 360 may control the fans 394₁-394₂ independently from one another. For example, in some embodiments, the fan 394₂ may be more active, or may be active for longer periods of time, than the fan 394₁, since the light source 320 may generate more heat than the PCB 355. In some embodiments, the controller 360 may be operative to activate, deactivate, or otherwise control the action of the fans 394₁-394₂ based on one or more conditions. For example, in this embodiment, the sensors 370 of the control system 330 further include an internal temperature sensor 376 which is configured to monitor one or more temperatures, each temperature being associated with a respective location of the lamp 300, and may more specifically be associated with a respective location of the cavity 319. The internal temperature sensor 376 may be configured to provide an indication of the one or more temperatures to the controller 360, which may allow the controller 360 to determine, based on the indication from the internal temperature sensor 376, whether any of the one or more temperatures exceed one or more respective allowed temperature thresholds. Thus, the controller 360 may be operative to control the action of the fans 394₁-394₂ based on whether or not one or more of the temperatures exceed their respective allowed temperature thresholds. In some embodiments, a speed of rotation of each of the fans 394₁-394₂ may be controlled by the controller 360 based on one or more of the temperatures and/or based on a difference between one or more of the temperatures and their respective allowed temperature threshold. In embodiments of the lamp 300 lacking an internal temperature sensor 376, the controller 360 may be configured to monitor a time of operation of the lamp 300 and may control the action of the fans 394₁-394₂ (such as, e.g., increasing their respective speed of rotation) based at least in part on the time of operations. Other factors which may be considered by the controller 360 when controlling the action of the fans 394₁-394₂ may include an intensity of the light source 320, an indication of the number of sensors 370 in use, an indication of whether the lamp 300 is receiving power from an external power source via the power connector 382, from the battery 388, or from both, and the like. Other factors may also be considered by the controller 360.

As discussed earlier, in this embodiment, the lamp 300 is detachably connected to the support 400 such that the lamp 300 can be disconnected and removed from the support 400 to be used independently from the support 400, and reconnectable to the support 400 to be used while carried by the support 400. More specifically, in this embodiment, the lamp 300 contains within the housing 312 all those components used for its operation, including the control system 330 and the light source 320, such that the lamp 300 may operate as a self-contained unit by the user (e.g., by holding it in his/her hands, for example, by one or more of the handles 318₁-318₃). As such, the lamp 300 may be relatively light and portable for use without the support 400. Additionally, the lamp 300 may be fitted for use with a variety of supports which may differ from the support 400, and may use one or more adaptors (not pictured) to facilitate use with such other supports.

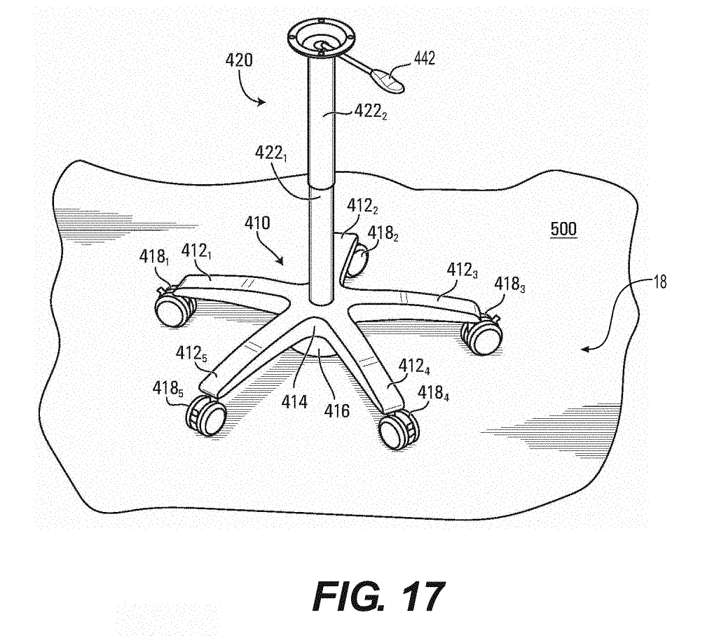

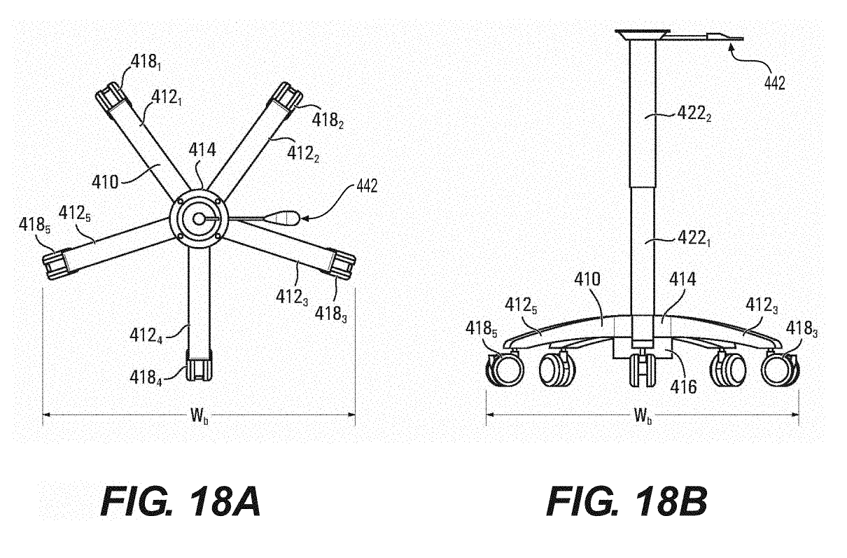

With additional reference to FIGS. 17 and 18, in this embodiment, the base 410 of the support 400 engages the surface 500 to support the phototherapeutic device 100 on the surface 500. The base 410 defines a width W_{b} of the support 400. The width W_{b} may have any suitable value. For instance, in some embodiments, the width W_{b} may be less than the opening provided by a standard doorframe, thereby allowing the phototherapeutic device 100 to be moved through such a doorway.

In this embodiment, the base 410 comprises a plurality of legs 412₁-412₅ extending radially from a core 414. In this example, the base 410 is rollable on the floor 18 to facilitate moving the phototherapeutic device 100. More particularly, in this example, each of the legs 412₁-412₅ is, at a distal end, connected to one of a plurality of rollers 418₁-418₅, which are configured to allow the phototherapeutic device 100 to roll on the floor 18. The base 410 may further comprise a lock to allow the user of the phototherapeutic device 100 to selectively prevent movement of the phototherapeutic device 100 by precluding rotation of one or more of the rollers 418₁-418₅. In some embodiments, the lock o10 applies simultaneously to all of the rollers 418₁-418₅, whereas in others the user may be able to apply the lock to individual ones of the rollers 418₁-418₅. The lock may be a mechanical lock, an electronically-controlled lock, or any other suitable lock. In this example, the base 410 comprises a counterweight 416 to prevent tipping of the phototherapeutic device 100 when the arm 430 carrying the lamp 300 is extended. In this case, the counterweight 416 is located in the core 414. In other cases, the counterweight 416 may be distributed through two or more of the legs 412₁-412₅.

Any other suitable configurations of the base 410 are possible. For example, some embodiments, the base 410 may comprise fewer than five legs, including three or four legs, or more than five legs, including six legs and more. In such embodiments, the number of rollers 418 may match the number of legs 412. As another example, in some embodiments, the base 410 may have a substantively flat bottom surface and no legs. In such embodiments, rollers 418 may jut out from the bottom surface or the support 400 may not have any rollers at all.

The post 420 extends upwardly from the base 410 and supports the arm 430. In this embodiment, the post 420 is implemented as a cylindrical shaft or other elongated cylinder-like member. In other embodiments, the post 420 may comprise an elongated member which may be a cuboid, a triangular prism, a pentagonal prism, or any suitably-shaped elongated member.

More particularly, in this embodiment, the post 420 is telescopic. The post 420 comprises a plurality of members 422₁-422₂ which are telescopically movable relative to one another, thereby allowing the user of the phototherapeutic device 100 to adjust a length of the post 420 and, by extension, a vertical position of the arm 430 and the lamp 300. In this embodiment, the members 422₁-422₂ may be configured such that a first one of the members 422₁-422₂ is configured to slideably move within a second one of the members 422₁-422₂. For example, the member 422₁ may have an elongated cylindrical shape having a radius r₁, and may be attached at a first end to the base 410; the member 422₂ may also have an elongated cylindrical shape having a radius r₂, and may be attached at a first end to the arm 430. In this embodiment, thus, the radius r₁ of the member 422₁ may be larger than the radius r₂ of the member 422₂, and members 422₁-422₂ may be positioned such that the second end of the member 422₁ surrounds the second end of the member 422₂, thereby allowing the second member 422₂ to move freely within the first member 422₁. In some embodiments, the roles of the members 422₁-422₂ may be reversed.

The phototherapeutic device 100 comprises an actuator 442 to telescopically move the 422₁-422₂ of the post 420 in a desired position relative to one another and fix them in that position. In some examples of implementation, the actuator 442 for the telescoping motion of the post 420 may be manually operated by the user of the phototherapeutic device 100. The actuator 442 may comprise a clamp or lock (not pictured) for securing the second member 422₂ at a particular position within the first member 422₁. In other examples of implementation, it may be possible for the user of the phototherapeutic device 100 to convey an indication of a desire to effect a change of the length of the post 420 via the user interface 351, which may relay the user's commands to the controller 360 which in turn may be configured to effect operation of the actuator 442 for changing the length of the post 420.

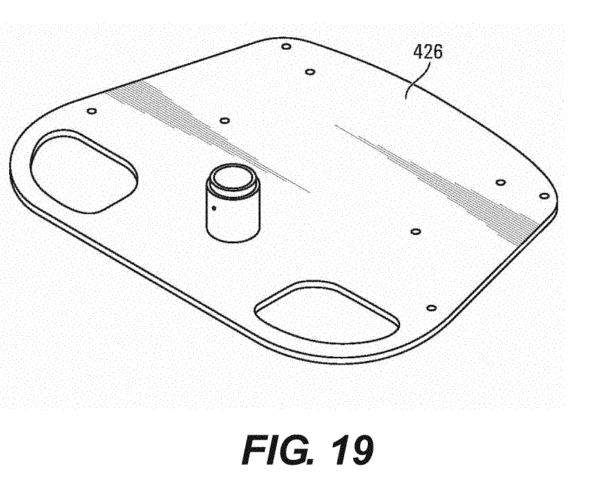

In this embodiment, and with additional reference to FIG. 19, the support 400 comprises a shelf 426 which is mounted to the post 420 and configured to support objects placed thereupon. The shelf 426 may comprise a connector for attachment to the post 420 and a substantively flat surface upon which objects may be placed for supporting. The surface may comprise one or more grooves or depressions which may be designed to hold specific categories of objects (such as a circular depression designed to act as a cup holder, or an elongated-rectangular-shaped depression designed to act as a pen tray). In some embodiments, one or more wall-like structures may be mounted on the surface of the shelf 426, configured so as to prevent the objects placed on the shelf from falling off one or more edges of the shelf.

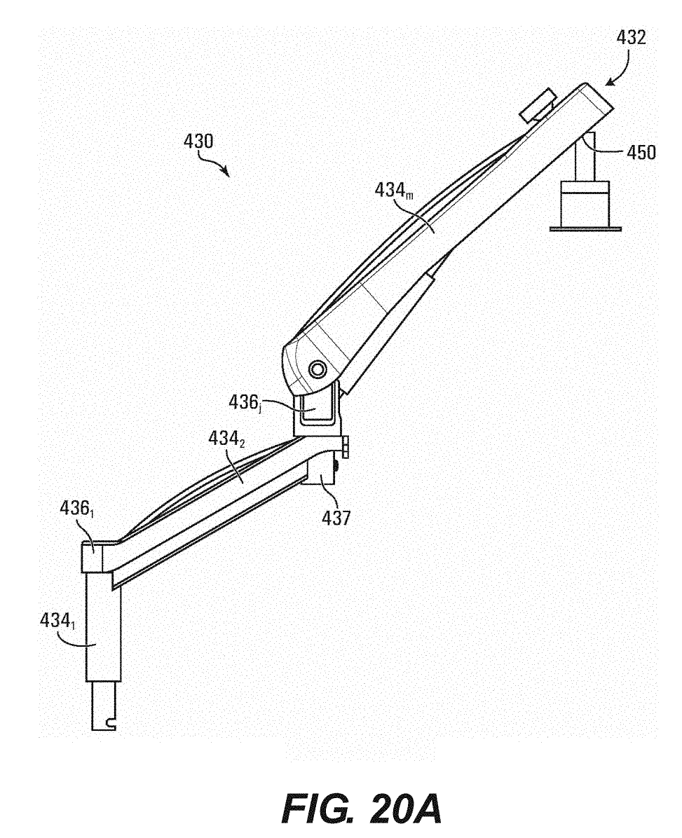

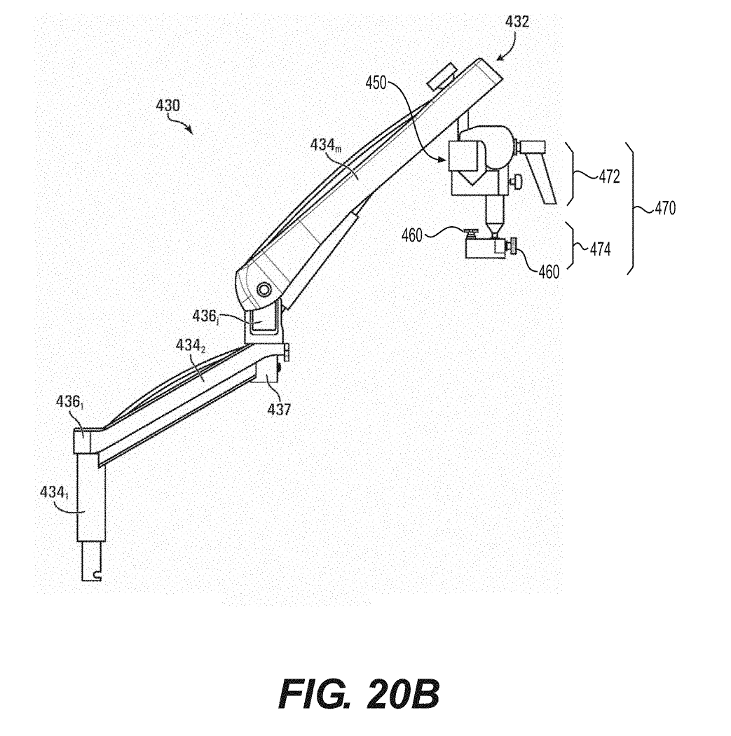

The arm 430 extends from the post 420 and supports the lamp 300. In this embodiment, the arm 430 is articulated to facilitate movement of the lamp 300. More particularly, with additional reference to FIG. 20A, in this embodiment, the arm 430 comprises a plurality of members 434₁-434ₘ which are interconnected and movable relative to one another. The members 434₁-434ₘ may be generally cylindrical and elongated, or may have any other suitable configuration.

The arm 430 comprises one or more joints 436₁-436ⱼ which are disposed between adjacent ones of the members 434₁-434ₘ of the arm 430 to allow them to move relative to one another. Thus, articulation of the arm 430 is implemented by the joints 436₁-436ⱼ which may allow the arm 430 to move in an articulated fashion. One or more of the joints 436₁-436ⱼ may comprise a joint lock 437 for locking a position of that joint and adjacent ones of the members 434₁-434ₘ between which it is disposed. For instance, the joint locks 437 may be operable manually by the user, and may be implemented, for example, as a set screw with a knob. In other embodiments, the joint locks 437 may be implemented as one or more electronic locks and may be controlled by the controller 360. In such embodiments, the user interface 351 may be configured to receive an indication from the user of a desire to cause one or more of the joint locks 437 to assume a locked or an unlocked state.

In this embodiment, the arm 430 comprises three members, namely the members 434₁, 434₂, and 434₃, and two joints, namely the joints 436₁, 436₂. The member 434₁ is connected to the post 420 at a first end and to the joint 436₁ at a second end. The member 434₂ is connected at a first end to the joint 436₁ and to the joint 436₂ at a second end. The member 434₃ is connected to the joint 436₂ at a first end and to the lamp-connecting system 450 at a second end.

Additionally or alternatively, in some embodiments, the arm 430 may comprise one or more actuators. The actuators may be located at any suitable location of the arm 430, including in one or more of the members 434₁-434ₘ and/or in the joints 436₁-436ⱼ. The actuators may be configured to effect changes in the position of one or more of the members 434₁-434ₘ, for example by causing one of the members 434₁-434ₘ to rotate around a point. In some embodiments, the point around which one of the members 434₁-434ₘ rotates may be one of the joints 436₁-436ⱼ, and more specifically may be one of the joints 436₁-436ⱼ to which the respective member 434₁-434ₘ is connected. The actuators may be implemented as electric motors, for example, or in any other suitable way.

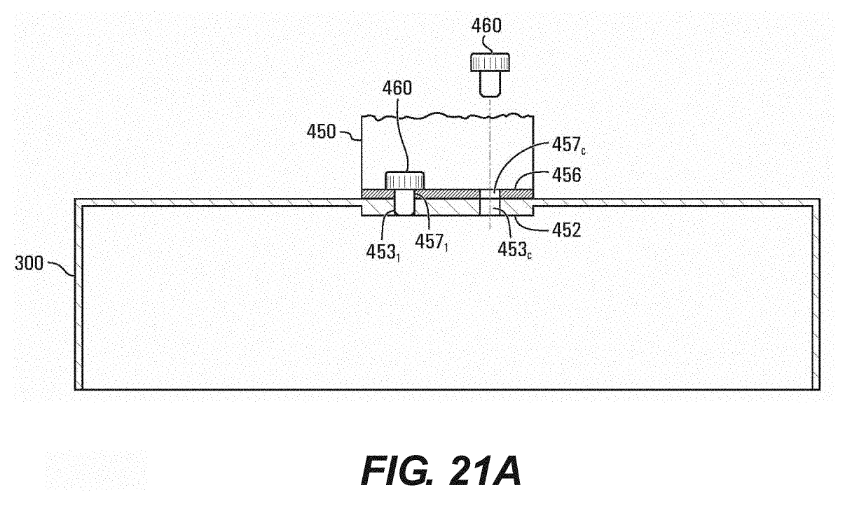

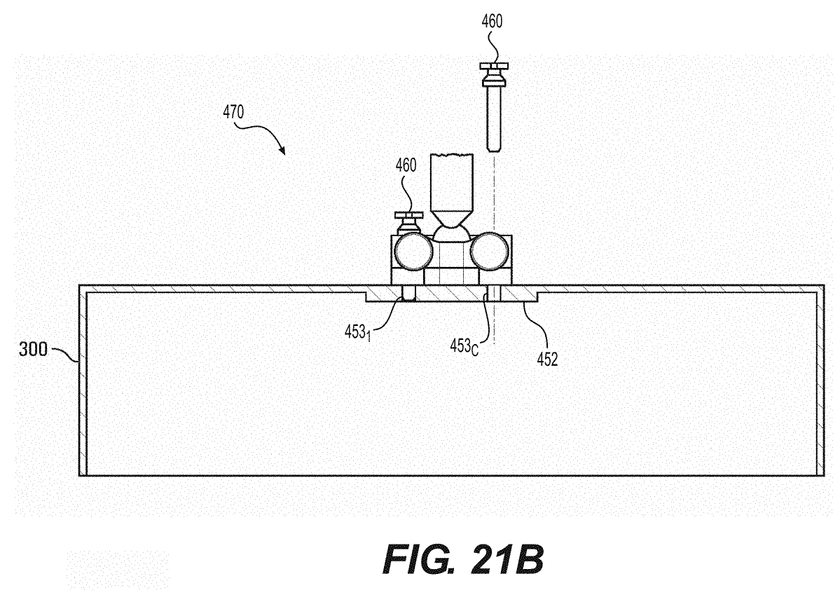

As discussed previously, in this embodiment, the lamp-connecting system 450 detachably connects the lamp 300 to the support 400. With additional reference to FIGS. 21A and B, the lamp-connecting system 450 comprises a connector 452 provided on the lamp 300 and a connector 456 provided on the support 400. In this embodiment, the connector 456 of the support 400 is provided on the arm 430.

In this embodiment, the lamp-connecting system 450 is configured such that the lamp 300 is toollessly disconnectable and removable from the arm 430 and toollessly reconnectable to the arm 430 to allow the user to toollessly disconnect and remove the lamp 300 from and toollessly reconnect the lamp 300 to the arm 430. Herein, “toollessly” means “manually without using any tool”. In other words, the lamp 300 is connectable to and disconnectable from the arm 430 manually without using any tool (e.g., a screwdriver or other tool) such that the user can connect the lamp 300 to the arm 430 manually without using any tool and can disconnect the lamp 300 from the arm 430 manually without using any tool. This makes mounting and removal of the lamp 300 to and from the support 400 rapid and convenient. In some embodiments, connection and disconnection of the lamp 300 from the arm 430 may require only a single action (e.g., releasing a clamp), whereas in other embodiments it may require a plurality of actions to be performed in short succession, or in a certain order (e.g. unscrewing fasteners then releasing a clamp).

More particularly, in this embodiment, the connector 452 of the lamp 300 may comprise a plurality of holes 453₁-453_{c}, and the connector 456 of the arm 430 comprise a plurality of holes 457₁-457_{c} that are disposed to be aligned with the holes 453₁-453_{c} of the connector 452 of the lamp 300 and to receive a fastener 460 to secure the lamp 300 and the arm 430 together. The fastener 460 is toollessly-operable to fasten and unfasten the lamp 300 to the arm 430 (e.g., a manually-operable screw having a knob).

The lamp-connecting system 450 may be configured in any other suitable way to allow the lamp 300 to be toollessly connectable to and toollessly disconnectable from the arm 430 in other embodiments. For example, in other embodiments, the lamp-connecting system 450 may alternatively be implemented as a clip, a clamp, a quick-connect mechanism, a magnetism-based retention system, a suction cup, or any suitable other implementation.

In other embodiments, the lamp-connecting system 450 may be configured such that the lamp 300 is connectable to and disconnectable from the arm 430 using a tool. The tool may be a fastening tool, such as a key (hex key, Allen key, or the like), a screwdriver, or any other suitable tool.

With reference to FIGS. 20B and 21B, an alternative example of the lamp-connecting system 450 is illustrated. Specifically, the lamp connecting system 450 has the aforementioned connector 452 of the lamp 300 and also has a clamp connector 470 which comprises a clamping portion 472 and a fastening portion 474 connected to the clamping portion 472. The clamping portion 472 may be any suitable clamp or retaining mechanism suitable for securing the clamp connector 470 to the distal end portion 432 of the arm 430. The clamping portion 472 may clamp directly to the distal end portion 432, such that opposing jaw elements of the clamping portion contact against and compress opposite sides of the distal end portion 432; alternatively, the distal end portion 432 may present a bar, hook, or other narrowed element upon which the clamping portion 472 may be clamped (such as illustrated in FIG. 20B). The fastening portion 474 may comprise one or more screws, bolts, or other fasteners, which may be inserted through the holes 453₁-453_{c} of the connector 452 of the lamp 300 and configured for securing the lamp 300 to the clamp connector 470, which in turn may be secured to the arm 430. In some cases, the clamping portion 472 is designed to be toollessly attached to and removed from the arm 430, whereas in other cases, a tool such as a screwdriver, ratchet, Allen key, or other suitable tool, may be required to attach and detach the clamp connector 470, and more specifically the clamping portion 472, to the arm 430.

In accordance with this embodiment, the lamp 300, the connecting system 450 and the support 400 may be separated from one another such that the lamp 300 may be transported independently from the connecting system 450 and the support 400.

As, in this embodiment, the arm 430 is articulated and the post 420 is telescopic, the lamp 300 has a range of movement Mₐ that may be greater than in other embodiments where the arm 430 is not articulated and/or where the post 420 is not telescopic. The range of movement Mₐ of the lamp 300 can be viewed as a volume delimited by all positions the lamp 300 can assume as a result of moving the arm 430 and the post 420. Additionally, the lamp-connecting system may comprise one or more joints, such as a ball-joint, which further increases the range of movement Mₐ of the lamp 300.

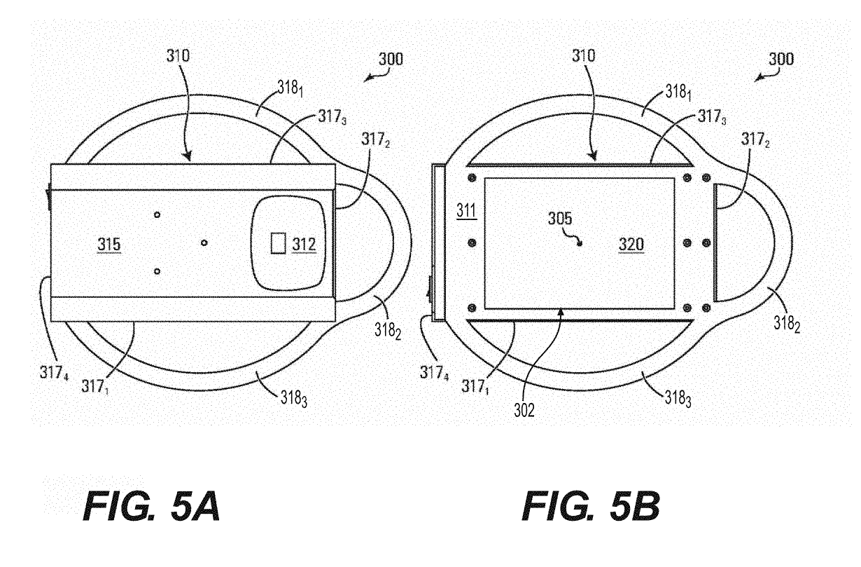

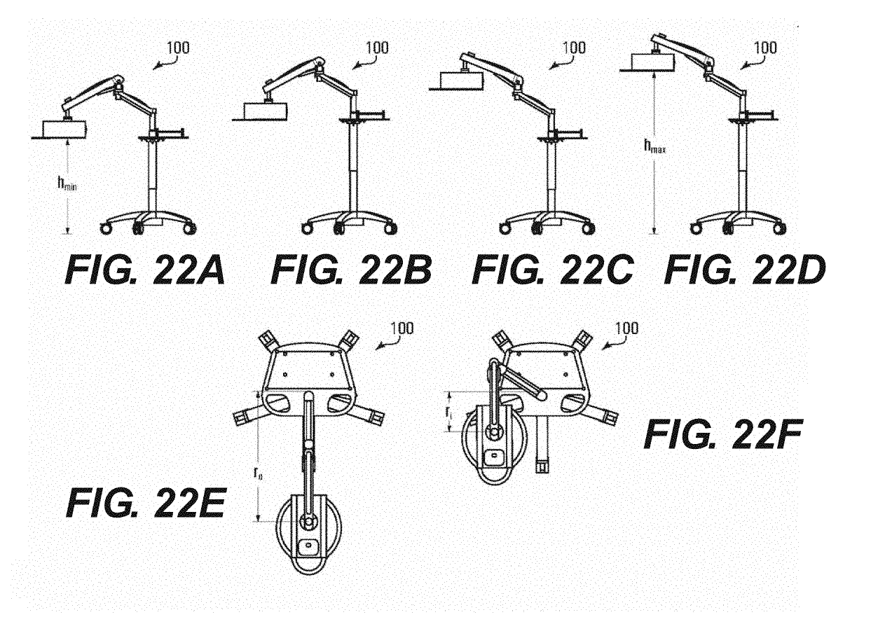

For example, in this embodiment, the range of movement Mₐ of the lamp 300 may be defined as a hollow-centre cylinder (or a three-dimensional donut-shape or torus-like shape). More specifically, and with reference to FIGS. 5A-B and additional reference to FIGS. 22A-F, the range of movement Mₐ of the lamp 300 may have a horizontal range and a vertical range. The lamp 300 attached to the arm 430 may be configured to rotate completely around an axis of rotation oriented along a longitudinal direction of the post 420 at a plurality of radii, such that a path traced by a reference point 305 of the lamp 300 over a complete rotation is a circle, each radius defining a circle of a different size (thereby defining a horizontal range), and may be configured to move up and/or down by both the articulated motion of the arm 430 and the telescopic motion of the post 420. The reference point 305 of the lamp 300 can be taken as a centroid of the light-emitting side 302 of the lamp 300.

With particular reference to FIGS. 5B, 22E, and 22F, from the perspective of the reference point 305 of the lamp 300, the horizontal range may be defined by a donut shape with an inner radius rᵢ and an outer radius rₒ. The inner radius rᵢ may be substantially equivalent to the distance from a centre of a horizontal cross-section of the post 420 to the fixed point 305 when the arm 430 is in a retracted position. Conversely, the outer radius rₒ may be substantially equivalent to the distance from the centre of the horizontal cross-section of the post 420 to the fixed point 305 when the arm 430 is in an extended position. Of course, the rᵢ is a smaller value than rₒ. In this embodiment, the horizontal range of the lamp 300 may comprise a continuous range of radii between rᵢ and rₒ, such that a complete rotation of the lamp 300 along the axis of rotation of the lamp 300 may trace a circle having a radius of any value between rᵢ and rₒ. In other embodiments, the horizontal range of the lamp 300 may comprise a defined finite plurality of radii between rᵢ and rₒ, such that a complete rotation of the lamp 300 along the axis of rotation of the lamp 300 may trace a circle having a radius of one of a defined finite plurality of values between rᵢ and rₒ.

With reference to FIGS. 22A-D, from the perspective of the reference point 305 of the lamp 300, the vertical range of the lamp 300 may be defined as a straight vertical line, ranging from a minimum height hₘᵢₙ to a maximum height hₘₐₓ. The minimum height hₘᵢₙ may be defined as the vertical distance between the surface 500 and the reference point 305 of the lamp 300 when the post 420 and the arm 430 are in retracted positions; conversely, the maximum height hₘₐₓ may be defined as the vertical distance between the surface 500 and the reference point 305 of the lamp 300 when the post 420 and the arm 430 are in extended positions.

The range of movement Mₐ of the lamp 300 may thus be defined as the product of the horizontal range and the vertical range of the lamp 300. For example, in some embodiments, rᵢ may be may be at least 10 cm, in some cases at least 13 cm, and in some cases even more, or may be any other suitable radius; rₒ may be at least 20 cm, in some cases at least 25 cm, and in some cases even more, or may be any other suitable radius; hₘᵢₙ may be at least 20 cm, in some cases at least 24 cm, and in some cases even more, or may be any other suitable height; and/or hₘₐₓ may be at least 30, in some cases at least 40 cm, and in some cases even more, or may be any other suitable height. As a result, the range of movement Mₐ may substantially cover a volume which may be at least 7000 cm³, in some cases at least 7250 cm³, in some cases at least 10000 cm³, in some cases at least 15000 cm³, in some cases at least 20000 cm³, and in some cases even more. The range of movement Mₐ of the lamp 300 may have any other suitable value in other embodiments.

In some embodiments, the counterweighting effect provided by the counterweight 416 may be determined based on the range of movement Mₐ of the lamp 300. More specifically, embodiments of the phototherapeutic device 100 having a larger range of movement Mₐ of the lamp 300 may have the counterweight 416 which is substantively heavier than in embodiments of the phototherapeutic device having a smaller range of movement Mₐ of the lamp 300. Additionally, the counterweighting effect may be sufficient to allow for the phototherapeutic device 100 to satisfy one or more tipping-prevention standards, such as the “IEC60601-1, Medical Electrical Equipment part 1: General requirements for basic safety and essential performance” sect. 9.4.2 (Instability-overbalance).

In some embodiments, the control system 330 may be configured to adjust a configuration of the support 400 based on a position of the lamp 300 relative to the subject 200 to ensure proper positioning of the lamp 300 for treatment purposes.

For example, in some embodiments, the controller 360 of the control system 330 may be operative to acquire information about the position of the lamp 300 relative to the subject 200, including via any of the sensors 370 (such as the distance measurer 372 or the monitoring sensor 374), via input from the user, and/or via any other suitable means. Based on the information acquired, the controller 360 may be configured to determine if the position of the lamp 300 relative to the subject 200 is appropriate for the phototherapy treatment procedure being executed. If the controller 360 determines that the position of the lamp 300 relative to the subject 200 is not appropriate, the controller 360 may be configured to alter the position of the lamp 300 by way of the telescopic motion of the post 420 (via the actuator 424) and/or by way of the articulation of the arm 430 (via the actuators 435) in order to move the lamp 300 closer to, further from and/or in a different orientation relative to the subject 200. Additionally or alternatively, such as in embodiments where the post 420 and/or the arm 430 do not comprise actuators, the control system 330 may be configured to output a message to the user (for example, via the display 346) indicating that the position of the lamp 300 relative to the subject 200 is not appropriate for the phototherapy treatment procedure being executed, and may additionally suggest corrective action, including changing the position of the subject 200 and/or changing the position of the lamp 300.

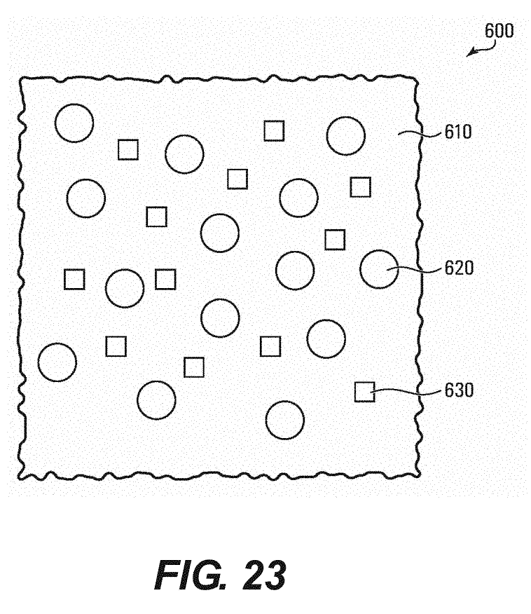

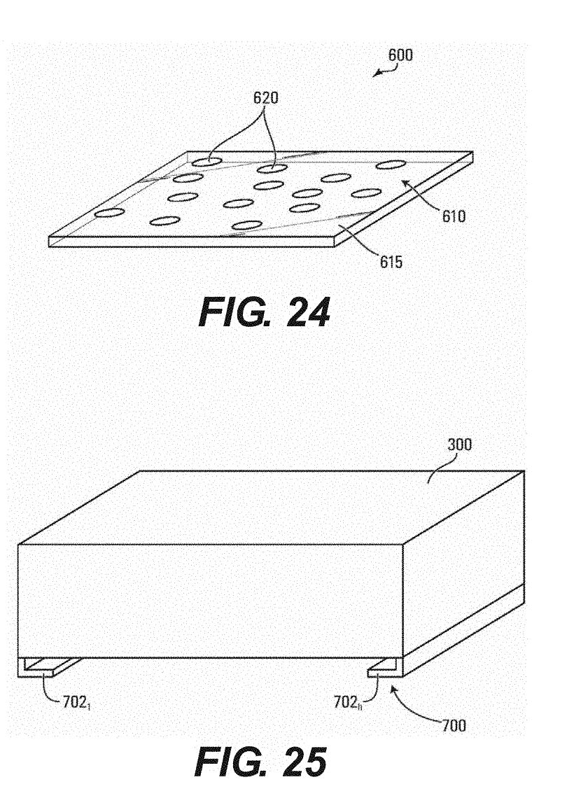

With additional reference to FIG. 23, in this embodiment, in order to implement the phototherapeutic treatment, the phototherapeutic device 100 may be used in combination with the photoactivatable composition 600 whereby the photoactivatable composition is illuminated by the light emitted by the lamp 300 to treat the subject 200. The photoactivatable composition 600 may comprises a carrier 610 and a photoactivatable agent 620 which may be dispersed in the carrier 610. In this some embodiments, the photoactivatable composition 600 may also comprise an oxygen-providing agent 630.

The carrier 610 is a medium carrying the photoactivable agent 620. In this embodiment, the carrier 610 is a fluid, a liquid or a semi-liquid (e.g., a gel). More particularly, in this embodiment, the carrier 610 may comprise a diluent, a solvent, a thickening agent, a cross-linker, a stabilizer, a surfactant, an initiator, an antimicrobial, or the like.

The photoactivatable agent 620 may be implemented in any suitable way. Examples of the photoactivable agent 620 in some embodiments include xanthenes derivatives, azo dyes, biological stains, carotenoids, fluorescent dyes or stains, biological dyes, histological dyes, chlorophyll dyes, methylene blue dyes, food colorings, naturally occurring photoactive agents, or the like.

In some implementations, the photoactivatable agent 620 is a xanthene dye. The xanthene dye may be fluorescein or eosin, or any other xanthene dye. In some implementations, the photoactivatable agent 620 is Eosin Y. In some other implementations, the photoactivatable agent 620 is Fluorescin. In some other implementations, the photoactivatable agent 620 is Eosin Y and Fluorescein. In some implementations, the photoactivatable agent 620 is Rose Bengal. In some other implementations, the photoactivatable agent 620 is Eosin Y and Rose Bengal. In some other implementations, the chromophore is Eosin Y, Rose Bengal and Fluorescein.

The oxygen-providing agent 630 may be implemented in any suitable way. Examples of the oxygen-providing agents 630 in some embodiments may include various peroxides, such as hydrogen peroxide, carbamide peroxide, urea peroxide, benzoyl peroxide, peroxy acids, and the like.

Examples of the photoactivatable composition 600 that may be used in various embodiments can be found, for example, in PCT/CA2009/001608; PCT/CA2009/001615; PCT/CA2010/001134; PCT/CA2013/000532; PCT/CA2013/000395; and PCT/US2013/058102.

In other embodiments, the carrier 610 of the photoactivatable composition 600 may be solid. In such embodiments, the carrier 610 comprises a solid material 615 allowing the light emitted by the phototherapeutic device 100 to enter the solid material and activate the photoactivatable agent 620. The solid material 615 may be transparent, translucent or otherwise allow at least some of the light emitted by the lamp 300 to enter it and possibly pass through it.

In some embodiments, the solid material 615 may be rigid. For instance, the photoactivatable composition 600 may be implemented as a panel. The panel may be made, for example, monomeric and/or polymeric materials, such as but not limited to 2-hydroxyethyl methacrylate (HEMA) or a polymer thereof. In other embodiments, the solid material 615 may be flexible. For instance, the photoactivatable composition 600 may be implemented as a sheet, such as a film or other thin member.

With additional reference to FIG. 25, in some embodiments, the phototherapeutic device 100 may comprise a holder 700 for holding the photoactivatable composition 600. The holder 700 is disposed to hold the photoactivatable composition 600 such that the photoactivatable composition 600 is maintained between the light-emitting side 302 of the lamp 300 and the subject 200.

More particularly, in this embodiment, the holder 700 is disposed to hold the photoactivatable composition 600 adjacent to the light-emitting side 302 of the lamp 300. In some examples of implementation, the holder 700 may be configured such that, when held by the holder 700, the photoactivatable composition 600 is contiguous to (i.e., in contact with) the light-emitting side 302 of the lamp 300. In other examples of implementation, the holder 700 may be configured such that, when held by the holder 700, the photoactivatable composition 600 is spaced apart from the light-emitting side 302 of the lamp 300. In such cases, the holder 700 may hold the photoactivatable composition 600 at a certain minimum distance from the light-emitting side 302 of the lamp 300 (e.g., to avoid overheating the photoactivable composition 600). In still further examples, the holder 700 may be adjustable, such that the distance between the light emitting side 302 and the photoactivatable composition 600 may be increased or decreased depending on the particular application. The manufacturer, distributor, or any other suitable party may also indicate a minimum distance of separation between the light-emitting side 302 and the photoactivatable composition 600, which may be any suitable value, such as 1 cm, 2 cm, 3 cm, 4 cm, 5 cm, 6 cm, 7 cm, 8 cm, 9 cm, 10 cm, or any other suitable distance.

In this embodiment, the holder 700 comprises a plurality of holding elements 702₁-702ₕ spaced apart from one another to engage the photoactivatable composition 600. In one example, the holding elements 702₁-702ₕ may include two or more tracks along which the photoactivatable composition 600 may be slidably positioned and held. In another example, the holding elements 702₁-702ₕ may comprise a plurality of hook-like elements which may hold the photoactivatable composition 600 at edge portions of the photoactivatable composition. Generally speaking, the holder 700 may be configured to minimize or avoid obscuring exposure of the photoactivatable composition 600 to the light emitted by the lamp 300. In some embodiments, the holder 700 may fasten the photoactivatable composition 600 adjacent to the light-emitting side 302 of the lamp 300 such that the holding elements 702₁-702ₕ may comprise one or more fastening elements, such as threaded fasteners, clips, magnets, hook-and-loop fasteners, or the like.

In some embodiments, the holder 700 may be an integral portion of the lamp 300. In other embodiments, the holder 700 may be detachably removable from the lamp 300. Alternatively, in other embodiments, the holder 700 may be an integral portion of the support 400, or may be detachably removable from the support 400.

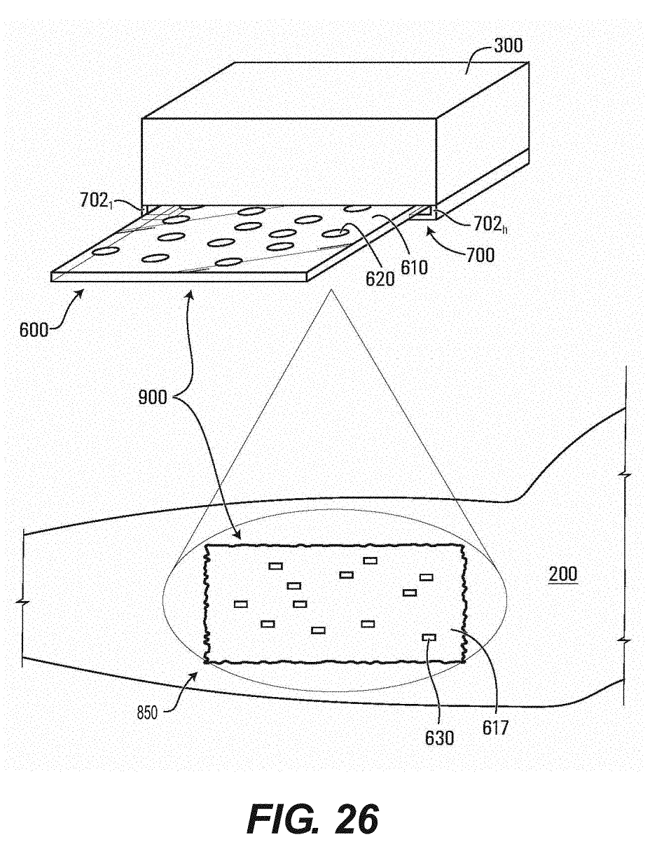

As an alternative, in some embodiments, with additional reference to FIG. 26, the photoactivatable agent 620 and the oxygen-providing agent 630 may be separated from each other, such that the photoactivatable agent 620 is included in the photoactivatable composition 600 while the oxygen-providing agent 630 is included in another photoactivatable composition 850. In this embodiment, the photoactivatable composition 850 comprises a carrier 617 in which is dispersed the oxygen-providing agent 630. In various examples of implementation, the carrier 617 may be implemented as discussed above in respect of the carrier 610 of the photoactivatable composition 600. The phototherapeutic device 100 is thus used in combination with a photoactivatable composition system 900 that comprises the photoactivatable composition 600 and the photoactivatable composition 800 which cooperate by being intersected by a common light transmission path.

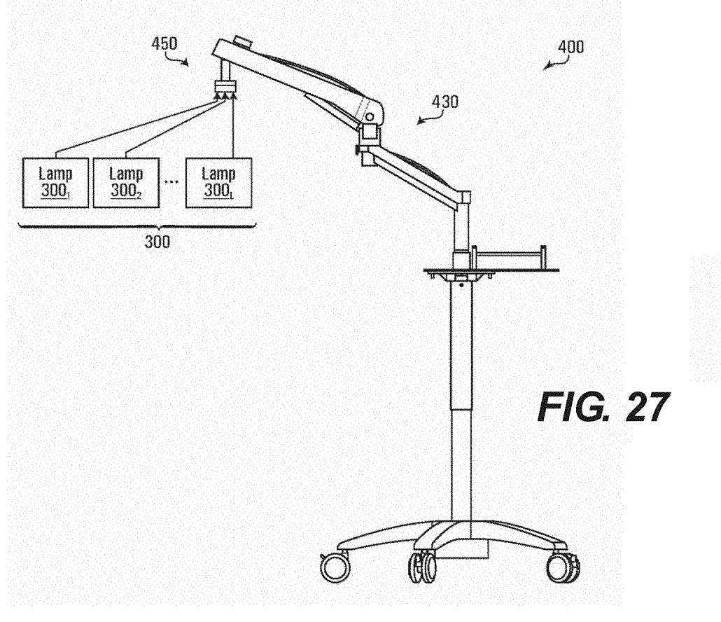

With additional reference to FIG. 27, in some embodiments of the phototherapeutic device 100, the lamp 300 may be one of a plurality of lamps 300₁-300_{L} that can be detachably connected to the support 400 by the lamp-connecting system 450 provided that the overall balance and stability of the lamp is conserved. In some examples, the lamps 300₁-300_{L} may have different shapes (e.g., different forms and/or different sizes) that may be configured for treatment of different areas of the subject 200 (e.g., different parts of the body of the subject 200). Additionally or alternatively, in some examples, the lamps 300₁-300_{L} may have different light emission characteristics, such as different intensities, different peak wavelengths, different combinations of wavelengths, or the like.

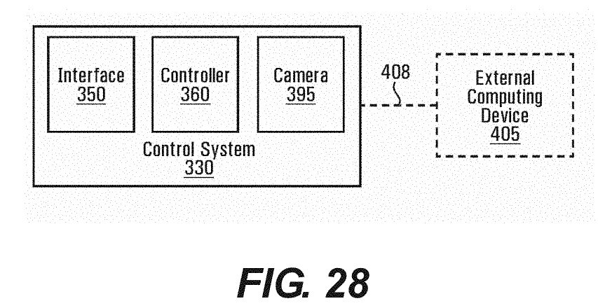

In some embodiments, with additional reference to FIG. 28, the control system 330 of the lamp 300 may comprise a camera 395 to capture images of the irradiated area 220 in order to acquire visual information about the irradiated area 220, which may include visual information about the treatment site 210 of the subject 200 and/or visual information about the photoactivatable composition 600 (and/or the photoactivatable composition 850 if applicable). Thus, the camera 395 may capture one or more images of the treatment site 210 of the subject 200 and/or of photoactivatable composition 600 (and/or the photoactivatable composition 850 if applicable) to be viewed and/or processed during the phototherapy treatment and/or subsequently.

In some examples of implementation, the camera 395 may be one of the sensors 370 of the control system 330, or may implement one or more of the sensors 370, such as the distance measurer 372 and/or the monitoring sensor 374. In other examples of implementation, the camera 395 may be separate from any other sensor that the control system 330 may comprise.

In some embodiments, the images captured by the camera 395 may be presented to the user, for example, on the display 346 of the user interface 351. Additionally or alternatively, in some embodiments, the images captured by the camera 395 may be stored in a storage device, such as, for example, in the memory portion 364 of the controller 360, or in an external storage device (not pictured) that is external to the phototherapeutic device 100. The stored images may then be retrieved at a later time (i.e., a time later than the moment at which they were captured) for viewing or processing via the phototherapeutic device 100 or via an external computing device 405, such as a personal computer, smartphone, or the like that is external to the phototherapeutic device 100. For instance, in some cases, the images may be retrieved during or soon after completion of the phototherapy treatment on the subject 200. In other cases, the images may be retrieved a significant period of time (e.g., one or more days, weeks or months) after completion of the phototherapy treatment on the subject 200.

The images are transmitted to the external computing device 405 over a communication link 408 (e.g., via the external device interface 358 of the phototherapeutic device 100). The communication link 408 may be wired, wireless, or partly wired and partly wireless and may be implemented in any suitable way. For example, in some cases, the communication link 408 may be implemented by a cable between the phototherapeutic device 100 and the external computing device 405. In other cases, the communication link 408 may be implemented by a data network (e.g., the Internet), a public telephony network (e.g., the PSTN), and/or a wireless network (e.g., a cellular network, a satellite network link).

In some embodiments, the camera 395 may capture images continuously over a certain period, as a video (i.e., moving images). In other embodiments, the camera 395 may capture images at specified moments in time, such as periodically or when a certain condition is fulfilled (e.g., at a beginning, midpoint, and end of the phototherapy treatment procedure on the subject 200). In some examples of implementation, the camera 395 may be associated with a microphone to capture sounds associated with the images (e.g., may be a video camera with audio capture capability).

In addition to and/or instead of being presented for viewing, in some embodiments, images captured by the camera 395 may be processed by the controller 360 to control operation of the lamp 300. For example, in some embodiments, images captured by the camera 395 may be processed to detect changes in the light emitted by the lamp 300, such as changes in intensity or peak or average wavelength, in the photoactivatable composition 600, such as changes in color, position, shape, reflectivity, transmissivity, and the like, or in the treatment site 210 of the subject 200, such as reddening. In response to detecting such changes, the controller 360 may perform an action relating to control of the lamp 300, such as change (e.g., decrease or increase) the intensity of light emitted by the lamp 300 (e.g., turn off the lamp 300), provide a notification on the user interface 351, or the like. In some examples, upon detecting such changes, or any other suitable changes, the camera 395 may be operative to acquire one or more images.

While in embodiments considered above the subject 200 is a human, the subject 200 treated by the phototherapeutic device 100 may be another animal or any other organism in other embodiments.

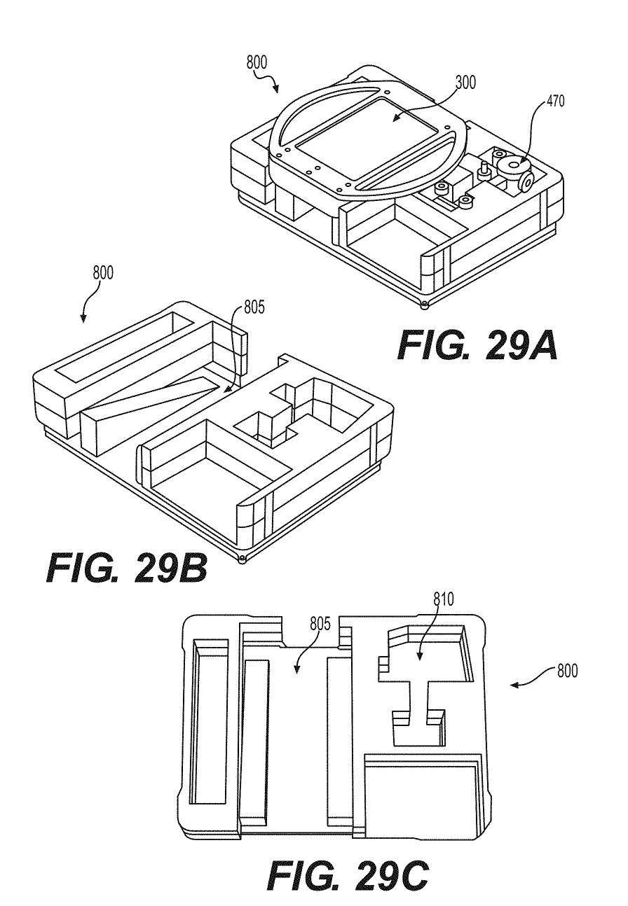

With reference to FIGS. 29A, 29B and 29C, an example of a casing for placement of the lamp 300 and the clamp connector 470 is illustrated. The casing 800 may be of various forms and shapes. In this embodiment, the casing 800 is substantially rectangular in shape and comprises an internal compartment 805 for accepting the lamp 300. The casing 800 may also comprise an internal compartment 810 for accepting the clamp connector 470. In some instances, the internal compartment which substantially corresponds to the shape of the lamp 300 and/or of the clamp connector 470 which is formed into a formable material such as, but not limited to, a polymeric foam. Preferably, the formable material and the compartments in the formable materials allow to protect the lamp 300 and/or the clamp connector 470 from moving during transportation and/or storage and from shocks. The casing may also comprise additional internal compartments and/or divisions for accepting other accessories for use in conjunction or combination with the lamp 300 and/or for the operation of the lamp 300. Such accessories, include, but are not limited to, a power supply, batteries, gloves, protective glasses, instructions, small tools, or the like.

The present disclosure also relates to kits for phototherapy.

In one embodiment, the kit comprises the phototherapeutic device 100 as well as instructions for use of the phototherapeutic device 100 in phototherapy. In some implementations of this embodiment, the kit may also comprise one or more of: the clamp connector 470, the power cable 384, the photoactivatable composition (600, 850), and spare light-generating elements 322₁-322_{L}.

In some other embodiments, the kit comprises the phototherapeutic device 100 as well as the clamp connector 470. In some implementations of this embodiment, the kit may also comprise one or more of: instructions for use of the phototherapeutic device 100 in phototherapy, the power cable 384, the photoactivatable composition (600, 850), and spare light-generating elements 322₁-322_{L}.

In some other embodiments, the kit comprises the lamp 300 as well as instructions for use of the lamp 300 in phototherapy. In some implementations of this embodiment, the kit may also comprise one or more of: the clamp connector 470, the power cable 384, the photoactivatable composition (600, 850), and spare light-generating elements 322₁-322_{L}.

In some other embodiments, the kit comprises the lamp 300 as well as the clamp connector 470. In some implementations of this embodiment, the kit may also comprise one or more of: instructions for use of the lamp 300 in phototherapy, the power cable 384, the photoactivatable composition (600, 850), and spare light-generating elements 322₁-322_{L}.

Any feature of any embodiment discussed herein may be combined with any feature of any other embodiment discussed herein in some examples of implementation.

Certain additional elements that may be needed for operation of certain embodiments have not been described or illustrated as they are assumed to be within the purview of those skilled in the art. Moreover, certain embodiments may be free of, may lack and/or may function without any element that is not specifically disclosed herein.

Although various embodiments and examples have been presented, this was for the purpose of describing, but not limiting, the invention. Various modifications and enhancements will become apparent to those skilled in the art and are within the scope of the invention, which is defined by the appended claims.

All documents referred to herein are incorporated by reference.

1. A phototherapeutic device comprising:
connection means; and
a lamp connected to the connection means, the lamp being detachably connected to the connection means such that the lamp is selectively disconnectable and removable from the connection means to be used independently from the connection means and reconnectable to the connection means to be used while supported by the connection means, the lamp comprising a light source;
wherein the light source emits light for phototherapy of a subject.

2. A phototherapeutic device as defined in claim 1, the lamp being toollessly disconnectable and removable from the connection means.

3. A phototherapeutic device as defined in claim 1, the lamp being toollessly reconnectable to the connection means.

4. A phototherapeutic device as defined in claim 1, the lamp having a range of movement to illuminate the subject's body.

5. A phototherapeutic device as defined in claim 1, the lamp further comprising:
a control system for controlling operation of the lamp;
a cooling system for cooling the lamp; and
a user interface for use by a user of the phototherapeutic device.

6. (canceled)

7. A phototherapeutic device as defined in claim 5, the control system comprising a controller operative to acquire information about position of the lamp relative to the subject.

8. A phototherapeutic device as defined in claim 7, the control system controlling emission of light by the lamp based on a distance between the lamp and the subject.

9. A phototherapeutic device as defined in claim 7, the control system further comprising a sensor providing information to the controller.

10. A phototherapeutic device as defined in claim 9, the information being information relating to the state of the lamp, and/or conditions of the environment surrounding the lamp, and/or progression of the phototherapy.

11.-14. (canceled)

15. A phototherapeutic device as defined in claim 5, wherein the cooling system comprises a first fan for directing airflow towards the light source; and a second fan for directing airflow towards the controller.

16. A phototherapeutic device as defined in claim 1, the phototherapeutic device being suitable for illumination of a photoactivatable composition.

17. A phototherapeutic device as defined in claim 16, the photoactivatable composition reacting to the light emitted by the light source of the phototherapeutic device.

18. A phototherapeutic device as defined in claim 16, the photoactivatable composition comprising a carrier and a photoactivatable agent dispersed in the carrier.

19. (canceled)

20. A phototherapeutic device as defined in claim 1, the phototherapeutic device further comprising a holder for holding a photoactivatable composition between a light-emitting side of the lamp and the subject.

21. A phototherapeutic device as defined in claim 1, the phototherapeutic device further comprising a camera for capturing images of an area illuminated by the light emitted by the lamp.

22. A phototherapeutic device as defined in claim 1, the light source comprising a plurality of light-generating elements collectively emitting the light for phototherapy.

23. A phototherapeutic device as defined in claim 22, wherein a first portion of the light-generating elements emits a first type of light and a second portion of the light-generating elements emits a second type of light.

24. (canceled)

25. A phototherapeutic device as defined in claim 24, wherein the first peak wavelength is between about 430 nm and 500 nm.

26. A phototherapeutic device as defined in claim 24, wherein the second peak wavelength is between about 400 nm and 500 nm.

27. A phototherapeutic device as defined in claim 22, wherein the first portion of the light-generating elements emits light at a first power density and the second portion of the light-generating elements emits light at a second power density.

28. A phototherapeutic device as defined in claim 27, wherein the first power density is less than about 200 mW/cm².

29. A phototherapeutic device as defined in claim 27, wherein the second power density is less than the first power density.

30.-36. (canceled)

37. A phototherapeutic device as defined in claim 1, wherein the light is for phototherapy of a treatment site of the subject.

38. A phototherapeutic device as defined in claim 37, wherein the treatment site is skin or a soft tissue.

39.-40. (canceled)

41. A phototherapeutic device as defined in claim 37, wherein the treatment site is an oral condition or a dental condition.

42.-43. (canceled)

44. A method for phototherapy of a treatment site on a subject, said method comprising:
a) applying a photoactivatable composition to the treatment site; and
b) exposing the applied photoactivatable composition to light emitted by a phototherapeutic device as defined in claim 1 to activate the photoactivatable composition;
wherein activation of the photoactivatable composition allows treatment of the treatment site.

45. A method for phototherapy of a treatment site on a subject, said method comprising:
a) mounting a photoactivatable composition to a phototherapeutic device as defined in claim 20; and
b) exposing the treatment site to light emitted by the phototherapeutic device to activate the photoactivatable composition;
wherein activation of the photoactivatable composition allows treatment of the treatment site.

46.-53. (canceled)
